SEQUENCE LISTING (25 sequences)

SEQ ID NO: 1 - RNA, 23 nt, Artificial Sequence
  Synthetic Polynucleotide
cuaccugcacuguuagcacuuug

SEQ ID NO: 2 - DNA, 23 bp, Artificial Sequence
  Synthetic Polynucleotide
ctacctgcactgttagcactttg

SEQ ID NO: 3 - RNA, 60 nt, Artificial Sequence
  Synthetic Polynucleotide
ccggcuaccugcacuguuagcacuuugaguuacuaccugcacucccgcacuuuguuuuug

SEQ ID NO: 4 - DNA, 60 bp, Artificial Sequence
  Synthetic Polynucleotide
ccggctacctgcactgttagcactttgagttactacctgcactcccgcactttgtttttg

SEQ ID NO: 5 - RNA, 132 nt, Artificial Sequence
  Synthetic Polynucleotide
accggcuaccugcacuguuagcacuuugaguuacuaccugccugcacucccgcacuuugaguuacuacugcacuguuagcacuguuagcacuuugaguuacuaccugcacucccgcacuuuguuuuuaauuc

SEQ ID NO: 6 - DNA, 132 bp, Artificial Sequence
  Synthetic Polynucleotide
accggctacctgcactgttagcactttgagttactacctgcctgcactcccgcactttgagttactactgcactgttagcactgttagcactttgagttactacctgcactcccgcactttgtttttaattc

SEQ ID NO: 7 - RNA, 225 nt, Artificial Sequence
  Synthetic Polynucleotide
ccggcuaccugcacuguuagcacuuugaguuacuaccugcacucccgcacuuugaguuacuaccugcacuguuagcacuuugaguuacuaccugcacucccgcacuuugaguuacuaccugcacuguuagcacuuugaguuacuaccugcacucccgcacuuugaguuacuaccugcacuguuagcacuuugaguuacuaccugcacucccgcacuuuguuuuug

SEQ ID NO: 8 - DNA, 225 bp, Artificial Sequence
  Synthetic Polynucleotide
ccggctacctgcactgttagcactttgagttactacctgcactcccgcactttgagttactacctgcactgttagcactttgagttactacctgcactcccgcactttgagttactacctgcactgttagcactttgagttactacctgcactcccgcactttgagttactacctgcactgttagcactttgagttactacctgcactcccgcactttgtttttg

SEQ ID NO: 9 - DNA, 106 bp, Artificial Sequence
  Synthetic Polynucleotide
ctgcgcgctcgctcgctcactgaggccgcccgggcaaagcccgggcgtcgggcgacctttggtcgcccggcctcagtgagcgagcgagcgcgcagagagggagtgg

SEQ ID NO: 10 - DNA, 241 bp, Artificial Sequence
  Synthetic Polynucleotide
gtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctc

SEQ ID NO: 11 - DNA, 1350 bp, Artificial Sequence
  Synthetic Polynucleotide
actagttattaatagtaatcaattacggggtcattagttcatagcccatatatggagttccgcctgcagggacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcggcgcgccttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagggcgcgccgggggggggggcgctgaggtctgcctcgtgaagaaggtgttgctgactcataccaggcctgaatcgccccatcatccagccagaaagtgagggagccacggttgatgagagctttgttgtaggtggaccagtcctgcaggagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcacgttatcgataccgtcgactagagctcgctgatcagtggggggtggggtggggcaggacagcaagggggaggattgggaagacaatagcagctgcagaagtttaaacgcatgc

SEQ ID NO: 12 - DNA, 141 bp, Artificial Sequence
  Synthetic Polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 13 - DNA, 5468 bp, Artificial Sequence
  Synthetic Polynucleotide
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccgtctcgaggtcgagaattcaaaaacaaagtgcgggagtgcaggtagtaactcaaagtgctaacagtgcaggtagtaactcaaagtgcgggagtgcaggtagtaactcaaagtgctaacagtgcaggtagtaactcaaagtgcgggagtgcaggtagtaactcaaagtgctaacagtgcaggtagtaactcaaagtgcgggagtgcaggtagtaactcaaagtgctaacagtgcaggtagccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtgaagactagttattaatagtaatcaattacggggtcattagttcatagcccatatatggagttccgcctgcagggacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcggcgcgccttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagggcgcgccgggggggggggcgctgaggtctgcctcgtgaagaaggtgttgctgactcataccaggcctgaatcgccccatcatccagccagaaagtgagggagccacggttgatgagagctttgttgtaggtggaccagtcctgcaggagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcacgttatcgataccgtcgactagagctcgctgatcagtggggggtggggtggggcaggacagcaagggggaggattgggaagacaatagcagctgcagaagtttaaacgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 14 - DNA, 106 bp, Artificial Sequence; identical to SEQ ID NO: 9
  Synthetic Polynucleotide

SEQ ID NO: 15 - RNA, 55 nt, Artificial Sequence
  Synthetic Polynucleotide
ccgguuagcacuuugacauggccacucgaguggccaugucaaagugcuaauuuug

SEQ ID NO: 16 - DNA, 56 bp, Artificial Sequence
  Synthetic Polynucleotide
caaaaattagcactttgacatggccactcgagtggccatgtcaaagtgctaaccgg

SEQ ID NO: 17 - DNA, 5299 bp, Artificial Sequence
  Synthetic Polynucleotide
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccgtctcgaggtcgagaattcaaaaattagcactttgacatggccactcgagtggccatgtcaaagtgctaaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtgaagactagttattaatagtaatcaattacggggtcattagttcatagcccatatatggagttccgcctgcagggacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcggcgcgccttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagggcgcgccgggggggggggcgctgaggtctgcctcgtgaagaaggtgttgctgactcataccaggcctgaatcgccccatcatccagccagaaagtgagggagccacggttgatgagagctttgttgtaggtggaccagtcctgcaggagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcacgttatcgataccgtcgactagagctcgctgatcagtggggggtggggtggggcaggacagcaagggggaggattgggaagacaatagcagctgcagaagtttaaacgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 18 - DNA, 5 bp, Artificial Sequence
  Synthetic Polynucleotide
agtta

SEQ ID NO: 19 - RNA, 5 nt, Artificial Sequence
  Synthetic Polynucleotide
aguua

SEQ ID NO: 20 - RNA, 23 nt, Artificial Sequence
  Synthetic Polynucleotide
caaagugcuaacagugcagguag

SEQ ID NO: 21 - DNA, 5786 bp, Artificial Sequence
  Synthetic
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccgtctcgaggtcgagaattcaaaaacaaagtgcgggagtgcaggtagtaactcaaagtgctaacagtgcaggtagtaactcaaagtgcgggagtgcaggtagtaactcaaagtgctaacagtgcaggtagtaactcaaagtgcgggagtgcaggtagtaactcaaagtgctaacagtgcaggtagtaactcaaagtgcgggagtgcaggtagtaactcaaagtgctaacagtgcaggtagccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtgaagactagttattaatagtaatcaattacggggtcattagttcatagcccatatatggagttccgcctgcagggacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcggcgcgccttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagggcgcgccgggggggggggcgctgaggtctgcctcgtgaagaaggtgttgctgactcataccaggcctgaatcgccccatcatccagccagaaagtgagggagccacggttgatgagagctttgttgtaggtggaccagtcctgcaggagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcacgttatcgataccgtcgactagagctcgctgatcagtggggggtggggtggggcaggacagcaagggggaggattgggaagacaatagcagctgcagaagtttaaacgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 22 - DNA, 5617 bp, Artificial Sequence
  Synthetic
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccgtctcgaggtcgagaattcaaaaattagcactttgacatggccactcgagtggccatgtcaaagtgctaaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcggtgaagactagttattaatagtaatcaattacggggtcattagttcatagcccatatatggagttccgcctgcagggacgtcgacggatcgggagatctcccgatcccctatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcggcgcgccttttaaggcagttattggtgcccttaaacgcctggtgctacgcctgaataagtgataataagcggatgaatggcagaaattcgccggatctttgtgaaggaaccttacttctgtggtgtgacataattggacaaactacctacagagatttaaagctctaatgtaagcagacagttttattgttcatgatgatatatttttatcttgtgcaatgtaacatcagagattttgagacacaacgtggctttccccccccccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagggcgcgccgggggggggggcgctgaggtctgcctcgtgaagaaggtgttgctgactcataccaggcctgaatcgccccatcatccagccagaaagtgagggagccacggttgatgagagctttgttgtaggtggaccagtcctgcaggagcataaagtgtaaagcctggggtgcctaatgagtgagctaactcacattaattgcgttgcgctcactgcccgctttccagtcgggaaacctgtcgtgcccgcccagtctagctatcgccatgtaagcccactgcaagctacctgctttctctttgcgcttgcgttttcccttgtccagatagcccagtagctgacattcatccggggtcagcaccgtttctgcggactggctttctacgtgtctggttcgaggcgggatcagccaccgcggtggcggcctagagtcgacgaggaactgaaaaaccagaaagttaactggcctgtacggaagtgttacttctgctctaaaagctgcggaattgtacccgcggccgatccaccggtcgccaccagcggccatcaagcacgttatcgataccgtcgactagagctcgctgatcagtggggggtggggtggggcaggacagcaagggggaggattgggaagacaatagcagctgcagaagtttaaacgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 23 - DNA, 56 bp, Artificial Sequence
  Synthetic
ccggttagcactttgacatggccactcgagtggccatgtcaaagtgctaatttttg

SEQ ID NO: 24 - RNA, 55 nt, Artificial Sequence; identical to SEQ ID NO: 15
  Synthetic

SEQ ID NO: 25 - RNA, 23 nt, Homo Sapiens
aaaagugcuuacagugcagguag

(19) United States
(12) Patent Application Publication
(10) Pub. No.: US 2023/0090989 A1
(43) Pub. Date: Mar. 23, 2023

(54) AAV-Mediated Targeting of MIRNA in the Treatment of X-Linked Disorders

(71) Applicants: RESEARCH INSTITUTE AT NATIONWIDE CHILDREN'S HOSPITAL, Columbus, OH (US); UNIVERSITY OF VIRGINIA PATENT FOUNDATION, Charlottesville, VA (US)

(72) Inventors: Kathrin Christine Meyer, Columbus, OH (US); Sanchita Bhatnagar, Free Union, VA (US); Jogender Tushir-Singh, Free Union, VA (US); Brian K. Kaspar, Westerville, OH (US); Shibi Likhite, Columbus, OH (US)

(21) Appl. No.: 17/800,371

(22) PCT Filed: Feb. 18, 2021
(86) PCT No.: PCT/US2021/018593

§ 371 Date: Aug. 17, 2022

Related U.S. Application Data
(60) Provisional application No. 62/978,285, filed on Feb. 18, 2020.

(51) Int. Cl. C12N 15/113; C12N 15/86; A61P 25/00

(52) U.S. Cl. CPC C12N 15/113; C12N 15/86; A61P 25/00; C12N 2750/14141

(57) ABSTRACT

The present disclosure relates to targeting of miRNA to activate expression of genes on the inactivated X chromosome. This gene therapy is useful for treating X-linked disorders, including Rett syndrome.

### CROSS REFERENCE TO RELATED APPLICATIONS AND INCORPORATION BY REFERENCE OF MATERIAL SUBMITTED ELECTRONICALLY

This application claims priority to U.S. Provisional Patent Application No. 62/978,285, filed on Feb. 18, 2020, which is incorporated by reference in its entirety.

This application contains, as a separate part of disclosure, a Sequence Listing in computer-readable form (Filename: 54983_SeqListing.txt; 36950 bytes—ASCII text file, created Feb. 18, 2021) which is incorporated by reference herein in its entirety.

### FIELD OF THE INVENTION

The present disclosure relates to targeting of miRNA to activate expression of genes on the inactivated X chromosome. This gene therapy is useful for treating X-linked disorders, including Rett syndrome.

### BACKGROUND

Rett syndrome (RTT) is an X linked neurodevelopmental disorder affecting approximately 1 in 10,000 girls. Patients exhibit vast mutation and disease heterogeneity. The onset of symptoms is typically characterized by the loss of previously achieved developmental milestones at 6-18 months of age with a progressive loss of motor function and cognitive function. Approximately 15,000 girls and women in the US and 350,000 patients worldwide suffer from RTT. RTT girls have a variety of problems that may include movement issues (apraxia, rigidity, dyskinesia, dystonia, tremors), seizures, gastrointestinal problems (reflux, constipation), orthopedic issues (contractures, scoliosis, hip problems), autonomic issues (breathing irregularities, cardiac problems, swallowing) as well as sleep problems and anxiety.

Nearly all RTT cases are caused by de novo loss-of-function mutations in the X-linked methyl-CpG binding protein 2 (MECP2) gene. Most RTT patients are females who are heterozygous for MECP2 deficiency, and due to random X chromosome inactivation approximately 50% of cells express the mutant MECP2 gene whereas the other 50% express wild-type MECP2.

In males, the symptoms of Rett syndrome are usually too severe to be viable. The disease phenotype in females is less severe thanks to the presence of a second X chromosome that does not carry a mutation in the MECP2 gene or another X-linked gene. During development, each cell randomly inactivates one of the two X chromosomes in females. Thus, females contain a mix of cells expressing either a healthy copy of the MECP2 gene or the mutated copy, depending on which X chromosome they inactivated.

RNA interference (RNAi) is a mechanism of gene regulation in eukaryotic cells that has been considered for the treatment of various diseases. RNAi refers to post-transcriptional control of gene expression mediated by microRNAs (miRNAs). Natural miRNAs are small (21-25 nucleotides), noncoding RNAs that share sequence homology and base-pair with 3′ untranslated regions of cognate messenger RNAs (mRNAs), although regulation in coding regions may also occur. The interaction between the miRNAs and mRNAs directs cellular gene silencing machinery to degrade target mRNA and/or prevent the translation of the mRNAs. The RNAi pathway is summarized in Duan (Ed.), Section 7.3 of Chapter 7 in Muscle Gene Therapy, Springer Science+Business Media, LLC (2010).

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac, et al. Journal of Translational Medicine 5, 45 (2007). Isolation of the AAV-B1 serotype is described in Choudhury et al., Mol. Therap. 24(7): 1247-57, 2016. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

There is a need for developing therapeutic approaches for treating X linked disorders such as Rett Syndrome.

### SUMMARY

The disclosure provides for a novel gene therapy approach for treating X-linked disorders, such as Rett Syndrome caused by X-linked gene loss-of-function mutations. Provided herein are polynucleotides and gene therapy vectors that target one or more miRNAs which are known to inactivate one or more gene(s) on the X chromosome. The polynucleotides and vectors disclosed herein are designed to inhibit miRNA(s) and thereby reactivate the wild type gene of interest on the inactivated X chromosome.

In various embodiments, the disclosure provides polynucleotides and vectors comprising a microRNA sponge cassette, wherein the microRNA sponge cassette comprises one or more nucleotide sequences that target one or more miRNA of interest. Targeting the miRNA of interest by the sponge results in binding and inactivation of the miRNA of interest, inhibition of expression of the miRNA of interest, and/or increasing the expression and/or activity of genes associated with X-linked disorders (X-linked genes).

“Target” as used herein, refers to binding, interacting or hybridizing to the miRNA of interest. “Targeting” a miRNA of interest results in or triggers degradation of the miRNA of interest or inhibits activity of miRNA of interest.

In various embodiments, the polynucleotide comprises one or more nucleotide sequences that target the microRNA of interest that are tandem multiplexes of perfectly or imperfectly complementary sequences to the microRNA of interest. In various embodiments, the nucleotide comprises one or more nucleotide sequences that target the microRNA of interest is at least at least 85% complementary to the sequence of the mature microRNA of interest, at least 90% complementary to the sequence of the mature microRNA of interest, at least 95% complementary to the sequence of the mature microRNA of interest, at least 96% complementary to the sequence of the mature microRNA of interest, at least 97% complementary to the sequence of the mature microRNA of interest, at least 98% complementary to the sequence of the mature microRNA of interest or at least 99% complementary to the sequence of the mature microRNA of interest.

The disclosure also provides for a polynucleotide comprising a microRNA sponge cassette, wherein the microRNA sponge cassette comprises at least 2 or more nucleotide sequences that target one or more miRNA of interest, at least 3 or more nucleotide sequences that target one or more miRNA of interest, at least 4 or more nucleotide sequences that target one or more miRNA of interest or at least 2 or more nucleotide sequences that target one or more miRNA of interest. In related embodiments, the microRNA sponge cassette comprises 2, 4, 6, or 8 repeats of a nucleotide sequences that target the microRNA of interest. In some embodiments, the sponge sequence is in the reverse orientation and therefore the sponge sequence is on complementary strand of the cassette.

In various embodiments, the disclosure provides for a polynucleotide comprising a microRNA sponge cassette, wherein the microRNA sponge cassette comprises one or more nucleotide sequences that target miR106a. For example, the polynucleotide comprises a nucleotide sequence that target a miRNA of interest comprising the nucleotide sequence of any one of SEQ ID NO: 1 or 2. In various embodiments, the polynucleotide comprises a microRNA sponge cassette comprising the nucleotide sequence of SEQ ID NO: 3, 4, 5, 6, 7 or 8. In various embodiments, the sponge cassette sequence is the RNA sequence of SEQ ID NO: 3, 5, or 7 or the DNA sequence of SEQ ID NO: 4, 6 or 8. The disclosure also provides for a polynucleotide comprising more than one microRNA sponge cassette, for example a polynucleotide comprising two microRNA sponge cassettes, three microRNA sponge cassettes, four microRNA sponge cassettes or five microRNA sponge cassettes. These microRNA sponge cassettes may target the same microRNA or different microRNAs.

The disclosure also provides a recombinant AAV (rAAV) having a genome comprising any of the polynucleotide sequences disclosed herein. In various embodiments, the rAAV genome comprises a U6 promoter. In alternative embodiments, the rAAV genome comprises a H1 promoter, 7SK or other polymerase 3 promoters. In any of these embodiments, the promoter is in the reverse orientation and therefore the U6 promoter is on complementary strand of the genome. In various embodiments, the rAAV genome further comprises a stuffer sequence. The stuffer sequence” as used herein, refers to a noncoding nucleotide sequence of variable length included in the vector (e.g. rAAV) to maintain the optimal packaging length of the vector construct. For example, the rAAV further comprises a stuffer sequence comprising the nucleotide sequence of SEQ ID NO: 11. In various embodiments, the rAAV genome comprises nucleotides 980 to 3131 of the nucleotide sequences of SEQ ID NO: 21. In other embodiments, the rAAV genome comprises nucleotides 980 to 2962 of the nucleotide sequences of SEQ ID NO: 22. In various embodiments, the vector is a serotype AAV1, AAV2, AAV3, AAV4, AAV5, AAV6, AAV7, AAV8, AAV9, AAVRH10, AAVRH74, AAV11, AAV12, AAV13, Anc80, or AAV7m8 or their derivatives.

The disclosure provides a rAAV particle comprising any of the rAAVs disclosed herein. The disclosure also provides a composition comprising any of the polynucleotides, rAAVs, or rAAV particles disclosed herein.

The disclosure provides methods of treating Rett syndrome comprising administering a therapeutically effective amount of any one of the rAAVs disclosed herein. The disclosure also provides for use of a therapeutically effective amount of any one of the rAAVs disclosed herein for the preparation of a medicament for treating Rett syndrome. The disclosure also provides a composition comprising any of the polynucleotides, rAAVs, or rAAV particles disclosed herein for the treatment of Rett syndrome.

The disclosure provides methods of activating expression of an X-linked gene comprising administering a therapeutically effective amount of any one of the rAAVs disclosed herein. The disclosure also provides for use of a therapeutically effective amount of any one of the rAAVs disclosed herein for the preparation of a medicament for activating expression of an X-linked gene. The disclosure also provides a composition comprising any of the polynucleotides, rAAVs, or rAAV particles disclosed herein for activating expression of an X-linked gene. In various embodiments, the X-linked gene is Methyl CpG binding protein 2 (MECP2).

The disclosure provides methods of treating an X-linked disorder comprising administering a therapeutically effective amount of any one of the rAAVs disclosed herein for the treatment of an X-linked disorder. The disclosure also provides for use of a therapeutically effective amount of the rAAV of any one of the rAAVs disclosed herein for the preparation of a medicament for treating an X-linked disorder. In related embodiments, the X-linked disorder is rett syndrome, hemophilia A, hemophilia B, Dent's disease 1, Dent's disease 2, DDX3X syndrome, Albinism-deafness syndrome, Aldrich syndrome, Alport syndrome, Anaemia (hereditary hypochromic), Anemia, (sideroblastic with ataxia), Cataract, Charcot-Marie-Tooth, Color blindness, Diabetes (insipidus, nephrogenic), Dyskeratosis congenita, Ectodermal dysplasia, Faciogenital dysplasia, Fabry disease, Glucose-6-phosphate dehydrogenase deficiency, Glycogen storage disease type VIII, Gonadal dysgenesis, Testicular feminization syndrome, Addison's disease with cerebral sclerosis, Adrenal hypoplasia, Granulomatous disease, siderius X-linked mental retardation syndrome, Agammaglobulinaemia Bruton type, Choroidoretinal degeneration, Choroidaemia, Albinism (ocular), fragile X syndrome, Epileptic encephalopathy (early infantile 2), Hydrocephalus (aqueduct stenosis), Hypophosphataemic rickets, Lesch-Nyhan syndrome (hypoxanthine-guanine-phosphoribosyl transferase deficiency), incontinentia pigmenti, Kallmann syndrome, paroxysmal nocturnal hemoglobinuria, Spinal muscular atrophy 2, Spastic paraplegia, Keratosis follicularis spinulosa, Lowe (oculocerebrorenal) syndrome, Menkes syndrome, Renpenning Syndrome, Mental retardation, Coffin-Lowry syndrome, Microphthalmia (Lenz syndrome), Muscular dystrophy (Becker, Duchenne and Emery-Dreifuss types), Myotubular myopathy, Night blindness, Norrie's disease (pseudoglioma), Nystagmus, Orofaciodigital syndrome, Ornithine transcarbamylase deficiency (type I hyperammonaemia), Phosphoglycerate kinase deficiency, Phosphoribosylpyrophosphate synthetase deficiency, Retinitis pigmentosa, Retinoschisis, Muscular atrophy/Dihydrotestosterone receptor deficiency, Spinal muscular atrophy, Spondyloepiphyseal dysplasia tarda, Thrombocytopenia, Thyroxine-binding globulin, McLeod syndrome.

Other features and advantages of the present disclosure will become apparent from the following detailed description. It should be understood, however, that the detailed description and the specific examples, while indicating preferred embodiments of the disclosure, are given by way of illustration only, because various changes and modifications within the spirit and scope of the disclosure will become apparent to those skilled in the art from this detailed description.

### BRIEF DESCRIPTION OF DRAWINGS

FIGS. 1A-1D show miRNAs as epigenetic regulators of XCI. (FIG. 1A) General schematic of CRISPR/Cas9 genome-wide screen. BMSL2 cells stably expressing Cas9 were transduced with the lentiCRISPRv2 library at a MOI of 0.2. Following puromycin selection, cells with Xi-Hprt expression were enriched in HAT selection media. (FIG. 1B) qRT-PCR for Hprt and MECP2 in BMSL2 expressing sgRNA for indicated miRNA. Results were normalized to a control (NS). (FIG. 1C) Allele-specific Taqman analysis for MECP2 in RTT-treated with control or miR106i and wild type (WT) neurons. Error bar, SD; *, p<0.01. The relative expression levels of miR106a in the cortex, spleen, liver and lung tissues for both male and female mice is shown in FIG. 1D.

FIGS. 2A-2D show miR106a inhibition reactivates known targets without affecting viability. (FIG. 2A) qRT-PCR for PAK5 and Ankrd52 (FIG. 2B) MTT assay for cells treated with NS or miR106 inhibitor or miR106a sgRNA. A black dotted line indicates seeding density at day 0. Error bar, SD. FIGS. 2C-2D shows the relative expression levels of MECP2 transcript in Patski cells and Rett neurons in the presence and absence of miR106a inhibitor (FIG. 2C) or miR106a SgRNA (FIG. 2D).

FIGS. 3A-3C show miR106a interacts with RepA. (FIG. 3A) Strategy to capture miR106a-RepA complex. (FIG. 3B) Competitive elution of RepA from miR106a-RepA complex using mismatch, perfect or imperfect complementary oligonucleotides. (FIG. 3C) qRT-PCR monitoring RepA in BMSL2 cells treated with miR106a mimic. Chr14 is a negative control. Error bar, SD; *, p<0.01.

FIGS. 4A-4C show miR106a does not regulate Xist transcription. (FIG. 4A) ChIP monitoring PolII binding on Xist and Gfp promoter in H4SV. (FIG. 4B) qRT-PCR for Xist expression in H4SV treated with either non-silencer (NS) or miR106a inhibitor. (FIG. 4C) qRT-PCR analysis for Xist in NS or miR106a depleted H4SV following actinomycin D treatment. GAPDH was used as a normalization control. Error bar, SD.

FIGS. 5A-5D Representative images and quantification of RNA FISH monitoring Xist in cells treated with control or miR106i. (FIG. 5A) Quantitation of Xist cloud area and Xist puncta staining using Image J (FIG. 5B). Error bar, SD; *, p<0.01. FIGS. 5C and 5D show miR106a-RepA free energy (FIG. 5C) and miR106a-RepA binding (FIG. 5D).

FIG. 6 shows miR106a inhibition by miRNA sponges. Renilla activity in BMSL2 expressing control or miR106sp or miR106sp and miR106i. Error bar, SD; *, p<0.01.

FIGS. 7A-7C show Loss of miR106a expresses MECP2 in RTT neurons to rescue phenotypic defects. (FIG. 7A) Taqman analysis for MECP2 in RTT and wild type (WT) neurons treated with control or LTV-miR106sp. (FIGS. 7B-C) Quantitative analysis of soma area (FIG. 7B) and number of neuronal branch points (FIG. 7C) in MAP2+ RTT neurons treated with control or LTV-miR106sp. Error bar, SD; *, p<0.01.

FIGS. 8A-8B show miR106a depletion rescues activity-dependent Ca2+ transients in RTT-neurons. (FIG. 8A) Representative images acquired during Ca2+ imaging showing control RTT (NS), miR106sp-treated (miR106sp) and wild type (WT) neurons. The warmth of the colors corresponds to Ca2+ concentration. (FIG. 8B) Ca2+ spikes (Left) and percent neuronal signaling (Right) in NS, miR106sp and WT neurons (n=100). Error bar, SD. *, p<0.01.

FIGS. 9A-9C show Mir106a inhibitor expresses Xi-linked MECP2 in primary mouse embryonic fibroblasts derived from XistΔ:Mecp2/Xist:Mecp2 mouse model. (FIG. 9A) Schematic of the breeding strategy for generating XistΔ:Mecp2/Xist:Mecp2. (FIG. 9B) Quantitative analysis of GFP+ nuclei isolated from the brain cells of mice by FACS analysis. (FIG. 9C) RT-PCR analysis monitoring expression of the Mecp2-Gfp and Mecp2 in female XistΔ:Mecp2/Xist:Mecp2-Gfp MEFs following treatment with control or mir106i. GAPDH was monitored as a loading control.

FIGS. 10A-10C show AAV9-mir106sp expresses Xi-linked MECP2 in the brain of XistΔ:Mecp2/Xist:Mecp2-Gfp mice. (FIG. 10A) Fluorescence analysis of brain cells in mice injected with AAV9-Gfp. (FIG. 10B) Fluorescence analysis for Mecp2-Gfp expression in the brain of XistΔ:Mecp2/Xist:Mecp2-Gfp mice injected with AAV9-Control and AAV9-miR106sp. (FIG. 10C) RT-PCR analysis monitoring expression of the Mecp2-Gfp and Mecp2 in female XistΔ:Mecp2/Xist:Mecp2-Gfp mice following treatment with control (Vehicle) or mir106sp. GAPDH was monitored as a loading control.

FIGS. 11A-11B show the viral vector, pAAV.miR106a Sponge.Stuffer.Kan map (FIG. 11A) and pAAV.miR106a Sponge.Stuffer.Kan vector sequence (FIG. 11B).

FIGS. 12A-12B show the viral vector, pAAV.miR106a shRNA.stuffer.Kan map (FIG. 12A) and pAAV.miR106a shRNA.stuffer.Kan vector sequence (FIG. 12B).

FIGS. 13A-13D show RNA sequences for mir106a sponge (sp 1) design 1 with 8 sponges (FIG. 13A, the sponge sequence is [SEQ ID NO: 7] (lower sequence) shown next to mouse miR106a-5p target sequence [SEQ ID NO: 20] above), mir106a sponge (sp 1) design 2 (FIG. 13B [SEQ ID NO: 3]), mir106a sponge (sp1) design 3 (FIG. 13C [SEQ ID NO: 5]), and an exemplary shRNA sequence that targets miR106a (FIG. 13D [SEQ ID NO: 15]).

FIGS. 14A-14D show AAV9-miR106sp rescues behavioral deficit in female ACpG-RTT mice. (FIG. 14A) Rotarod performance for AAV9-control (circle) and AAV9-miR106sp (square)-injected female mice at 4- and 7-weeks. Day 1 represents base-line performance; maximum time is 300 seconds (Dashed line). (FIGS. 14B-D) Barnes maze performance at 7-weeks plotted as mean latency (FIG. 14B), mean velocity (FIG. 14C), and total distance travelled (FIG. 14D) by AAV9-control (circle) and AAV9-miR106sp (square)-injected female mice. n=3. Error bar, SD. *, p<0.01.

FIGS. 15A-15C show survival up to 250 days of age as well as, phenotypic scoring and rotarod performance of AAV9.miR106sp treated animals (mice) at 16 weeks of age. (FIG. 15A) Survival curve of AAV9-Control (empty viral particle) treated animals versus healthy littermates (genetic control) and AAV9-miR1065p treated animals shows strong improvement in survival with no deaths up to 250 days. (FIG. 15B) A graph demonstrating improvement in phenotypic scoring of treated animals up to 21 weeks of age compared to AAV9-Control treated animals. (FIG. 15C) Rotarod performance of AAV9-miR106sp treated animals at 16 weeks of age compared to AAV9-control or untreated animals demonstrates drastically improved ability to hold on to a spinning wheel measured in seconds (p<0.0001) presented as a heat map (upper panel) for individual animals in each group and quantified in a graph (bottom panel). Error bars indicate SEM.

### DETAILED DESCRIPTION

The present disclosure provides for a novel gene therapy approach for treating X-linked disorders, such as Rett Syndrome caused by X-linked gene loss of function mutations. For example, Rett Syndrome is an X-linked disorder affecting predominantly females as the consequences of a heterozygous loss of function mutation in the X-linked methyl CpG binding protein 2 (MECP2) gene. The gene therapy approach disclosed herein takes advantage of the fact that each cell that expresses the mutated form of MeCP2, also contains the natural backup copy of the gene on the inactivated X chromosome. Thus, reactivation of parts of the silenced chromosome re-express the healthy gene.

Provided herein are gene therapy vectors that target miRNA which are known to inactivate genes on the X chromosome. The gene therapy disclosed herein is designed to inhibit miRNA and thereby reactive the wild type gene of interest on the inactivated X chromosome. For example, miRNA106a is known to inactivate a portion of the X chromosome including the MECP2 gene, and gene therapy methods targeting miRNA106a will reactivate expression of genes in this cluster on the X chromosome.

### MicroRNAs

MicroRNAs (miRNAs) are single-stranded RNAs of ˜22 nucleotides that mediate gene silencing at the post-transcriptional level by pairing with bases within the 3′ UTR of mRNA, inhibiting translation or promoting mRNA degradation. A seed sequence of 7 bp at the 5′ end of the miRNA targets the miRNA; additional recognition is provided by the remainder of the targeted sequence, as well as its secondary structure.

### MiRNA Sponges

To achieve efficient miRNA inhibition in vivo, miRNA loss-of-function “sponges” were designed. In various embodiments, the disclosure provides a nucleic acid or nucleotide cassette that acts as a microRNA sponge that competitively inhibit one or more mature miRNAs in vivo. The miRNA sponge is a nucleotide sequence that comprises multiple target sites which are complementary to a miRNA of interest. These target sites are designed to bind to the miRNA of interest, which in turn causes degradation of the targeted miRNA.

For example, provided herein are microRNA sponges designed to target miRNA106a which is associated with Rett Syndrome and/or other X-linked disorders. Targeting the sponge to miRNA106a will induce degradation of miRNA106a and therefore interferes with miRNA106a-induced X chromosome silencing and thereby reactivates genes on the X chromosome, for e.g., the MECP2 gene.

“miRNA of interest” as used herein, refers to one or more miRNAs to which the microRNA sponge or small RNA binds to and inactivates or prevents the expression of (i.e. the miRNA which the miRNA sponge targets). In various embodiments, the sponge may target multiple microRNAs of interest.

In various embodiments, the sponge cassette may comprise tandem multiplexes of either perfectly or imperfectly complementary nucleotide sequence that bind to the miRNA of interest which “sop up” any miRNA of interest. In related embodiments, imperfectly complementary nucleotide sequences that target the microRNA of interest can result in “bulges” of the sponge cassette. “Bulge” refers to a secondary nucleic acid structure which a sponge cassette can form.

The sponge cassettes comprise one or more sequences that target or bind to a miRNA of interest. The sponge cassette may comprise multiple identical nucleic acid or nucleotide sequences that target single miRNA of interest, or the sponge cassette may comprise multiple different sequences that target a single miRNA of interest. Alternatively, the sponge cassette may comprise multiple different nucleotide sequences that target one or more miRNA of interest.

In addition, the microRNA sponge cassette comprises one or more nucleotide sequences that targets the miRNA of interest. In various embodiments, the one or more nucleotide sequences that target the microRNA of interest is at least at least 85% complementary to the mature microRNA of interest sequence, at least 90% complementary to the mature microRNA of interest sequence, at least 95% complementary to the mature microRNA of interest sequence, at least 96% complementary to the mature microRNA of interest sequence, at least 97% complementary to the mature microRNA of interest sequence, at least 98% complementary to the mature microRNA of interest sequence or at least 99% complementary to the mature microRNA of interest sequence.

In various embodiments, the microRNA sponge cassette comprises at least 2 or more nucleotide sequences that target one or more miRNA of interest, at least 3 or more nucleotide sequences that target one or more miRNA of interest, at least 4 or more nucleotide sequences that target one or more miRNA of interest or at least 2 or more nucleotide sequences that target one or more miRNA of interest.

In various embodiments, the sponge cassettes comprises a nucleotide sequence that bind 1, 2, 3, 4, 5, 6, 7, 8, 9 or 10 different miRNAs of interest. In certain embodiments, the sponge cassette comprises one or more nucleotide sequences that target one miRNA of interest. In certain embodiments, the sponge cassette comprises one or more nucleotide sequence that target two different miRNAs of interest or three different miRNAs of interest or four different miRNAs of interest or five different miRNAs of interest.

In various embodiments, the sponge cassette comprises multiple copies or “repeats” of the nucleotide sequence that targets the miRNA of interest wherein the “sops up” any miRNA of interest present at the site where the vector is expressed. In various embodiments, one or more sponge cassettes may contain 1, 2, 3, 4, 5, 6, 7, 8, 9 or 10 repeats of the nucleotide sequence that targets the miRNA of interest. In certain embodiments, the sponge cassette contains 2 repeats of the nucleotide sequence that targets the miRNA of interest. In certain embodiments, the sponge cassette contains 4 repeats of the sequence that targets the miRNA of interest. In certain embodiments, the sponge cassette contains 6 repeats of the nucleotide sequence that targets the miRNA of interest. In certain embodiments, the sponge cassette contains 8 repeats of the sequence that targets the miRNA of interest.

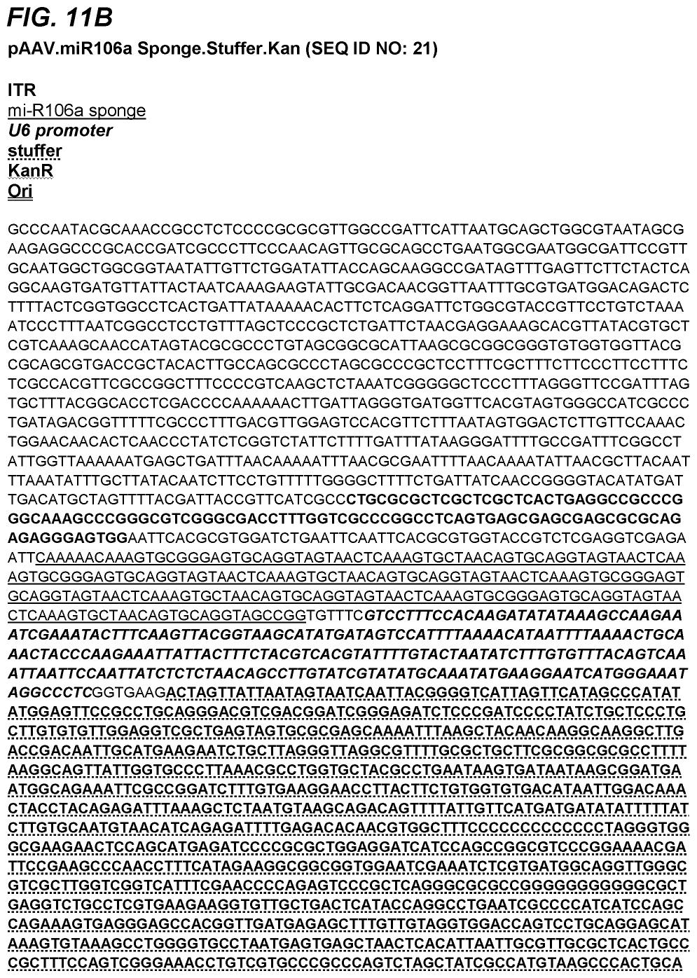

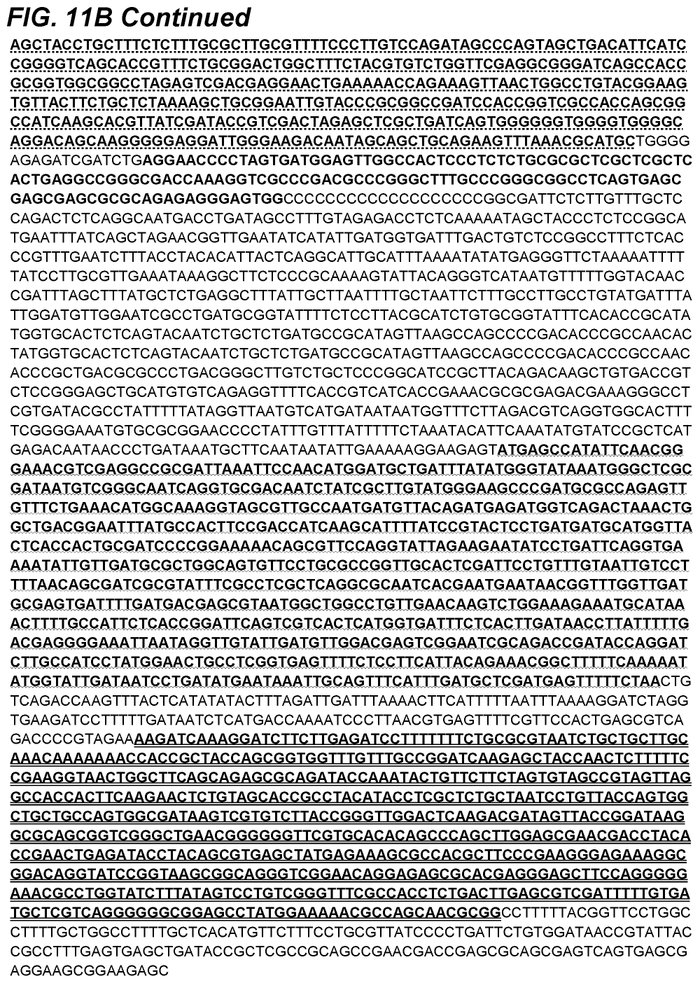

In some embodiments, the rAAV also may contain a stuffer sequence. The stuffer sequence is included in the vector to maintain optimal packaging length of the viral vector construct. The length of the stuffer sequence depends on the length of the sponge cassette. For example, the vectors contain a stuffer sequence that ranges in length from 1000 to 1500 base in length, or ranges from 500 to 2000 bases in length or ranges in 100 to 1000 bases in length. Exemplary stuffer sequences are 100 bases in length, or 200 bases in length, or 300 bases in length, or 400 bases in length, or 500 bases in length, or 600 bases in length, or 700 bases in length, or 800 bases in length, or 900 bases in length, or 1000 bases in length, or 1100 bases in length, or 1200 bases in length, or 1300 bases in length, or 1400 bases in length, or 1500 bases in length, or 1600 bases in length, or 1700 bases in length, or 1800 bases in length, or 1900 bases in length, or 2000 bases in length. To date, none of the FDA approved stuffer sequences are readily available. There are, however, several plasmid backbones that are approved by FDA for the human administration. Small DNA fragments were picked from these plasmids which do not correspond to any essential DNA sequences necessary for selection and replication of the plasmid or the elements of the transcriptional units. Exemplary plasmid backbones are listed in Table 1 and shown in FIGS. 11A-11B. The DNA elements from different plasmids were arranged in tandem to generate a complete, 1350 bp stuffer sequence (SEQ ID NO: 11).

miRNA106a

A large-scale loss-of-function screen identified miRNAs that when inhibited allow reexpression of the MECP2 gene from the inactivated X chromosome. Based on the results from cellular models, miRNA sponges were designed to inhibit microRNA 106a (also referred to as “miRNA106a or “miR106a”) and vectors were designed to deliver this sponge in vivo. In various embodiments, the disclosure provides vectors such as recombinant AAV vectors (rAAV) comprising one or more microRNA sponge cassettes targeting miRNAs of interest such as miR106a. MiR106a is encoded by miR106a-363 cluster on the X chromosome. Analysis of publicly available miR106a-CLIP data revealed multiple miR106a seed region in XistRNA. Up-regulation of miR-106a is positively correlated with tumor metastasis in patients with gastric cancer. MiR106a knockout mice are viable and show no phenotype. MiR106a is highly expressed in mouse brain cortex.

In an exemplary embodiment, the sponge cassette comprises sequences that target miRNA106a (miR106a). The sequence of mouse miRNA106a-5p is provided in SEQ ID NO: 20 and the sequence of human miRNA106a-5p is provided in SEQ ID NO: 25. Exemplary sequences that target the miRNA106a are set out as SEQ ID NO: 1 and SEQ ID NO: 2. The miRNA106(a) sponge sequence may comprise one or more copies of SEQ ID NO: 1 or 2 or one or more copies of a sequence that is at least 90% identical to SEQ ID NO: 1 or 2. The copies of SEQ ID NO: 1 or 2 may be separated by a spacer sequence, such as AGTTA (SEQ ID NO: 18) or AGUUA (SEQ ID NO: 19), in between the copies of any one of SEQ ID NO: 1 or 2. In various embodiments, the miR106a sponge is the nucleotide sequence as shown in SEQ ID NO: 3, 4, 5, 6, 7 or 8 or within the AAV genome of SEQ ID NO: 21 (nucleotides 1144-1368). In various embodiments, the miR106a sponge cassette sequence comprises the nucleotide sequence set forth in any one of SEQ ID NO: 1 or 2, or a variant thereof comprising at least about 90% identity to the nucleotide sequence set forth in any one of SEQ ID NO: 1 or 2. In any of these embodiments, the sponge sequence is in the reverse orientation and therefore the sponge sequence is on complementary strand of the cassette.

### MiRNA Small RNA

As set out herein above, the disclosure includes the use of inhibitory RNAs to be used alone or in conjunction with the miRNA sponges described herein to further reduce or inhibit miRNA of interest activity and/or expression. Thus, in some aspects, the products and methods of the disclosure also comprise short hairpin RNA or small hairpin RNA (shRNA) to affect miRNA of interest expression (e.g., knockdown or inhibit expression or inactivate one or miRNA(s) of interest). A short hairpin RNA (shRNA/Hairpin Vector) is an artificial RNA molecule (nucleotide) with a tight hairpin turn that can be used to silence target gene expression via RNA interference (RNAi). ShRNA is an advantageous mediator of RNAi in that it has a relatively low rate of degradation and turnover, but it requires use of an expression vector. Once the vector has transduced the host genome, the shRNA is then transcribed in the nucleus by polymerase II or polymerase III, depending on the promoter choice. The product mimics pri-microRNA (pri-miRNA) and is processed by Drosha. The resulting pre-shRNA is exported from the nucleus by Exportin 5. This product is then processed by Dicer and loaded into the RNA-induced silencing complex (RISC). The sense (passenger) strand is degraded. The antisense (guide) strand directs RISC to mRNA that has a complementary sequence. In the case of perfect complementarity, RISC cleaves the mRNA. In the case of imperfect complementarity, RISC represses translation of the mRNA. In both of these cases, the shRNA leads to target gene silencing. In some aspects, the disclosure includes the production and administration of an AAV vector expressing one or more miRNA target antisense sequences via shRNA. The expression of shRNAs is regulated by the use of various promoters. The promoter choice is essential to achieve robust shRNA expression. In various aspects, polymerase II promoters, such as U6 and H1, and polymerase III promoters are used. In some aspects, U6 shRNAs are used.

In various embodiments, the disclosure provides vectors comprising one or more small RNA targeting one or more miRNAs of interest. In various embodiments, the small RNAs are designed to target one or more miRNAs of interest associated with X-linked disorders (e.g. Rett Syndrome). In various embodiments, the binding of the small RNA to the microRNA of interest will induce its degradation and therefore interferes with X chromosome silencing.

In various embodiments, the terms “small RNA” or “small RNAs” as used herein, refer to small RNAs known to trigger RNAi processes in mammalian cells, including short (or small) interfering RNA (siRNA), and short (or small) hairpin RNA (shRNA) and microRNA (miRNA). Small RNAs are <200 nucleotides in length, and are typically non-coding RNA molecules.

In various embodiments, the small RNA is a polynucleotide comprising a nucleotide sequence that targets one or more microRNAs of interest. In related embodiments, the small RNA comprises a nucleotide sequence that bind 1, 2, 3, 4, 5, 6, 7, 8, 9 or 10 different miRNAs of interest. In certain embodiments, the small RNA comprises one or more nucleotide sequences that target one miRNA of interest. In certain embodiments, the small RNA comprises one or more nucleotide sequence that target two different miRNAs of interest or three different miRNAs of interest or four different miRNAs of interest or five different miRNAs of interest.

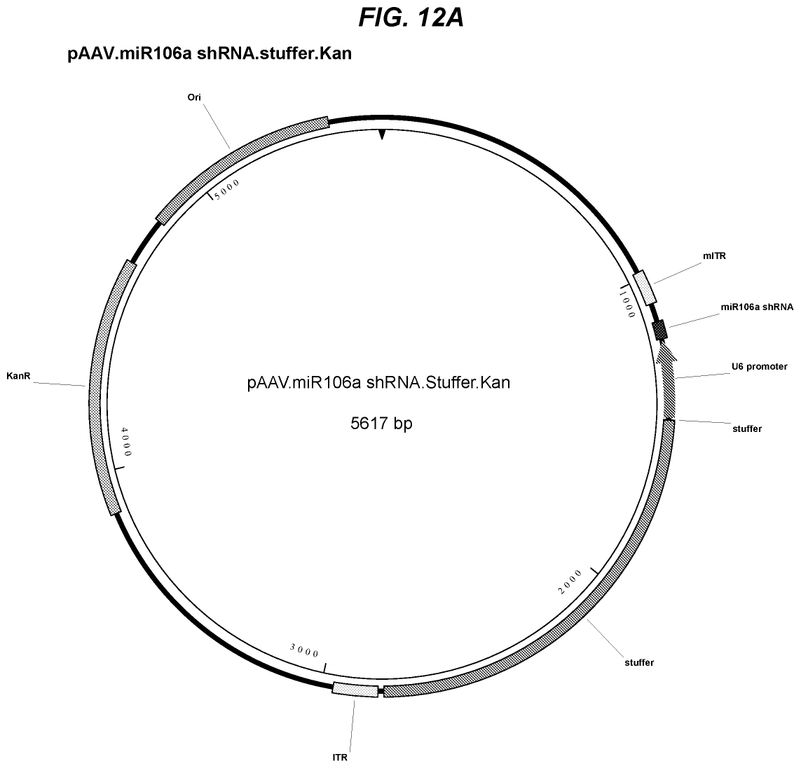

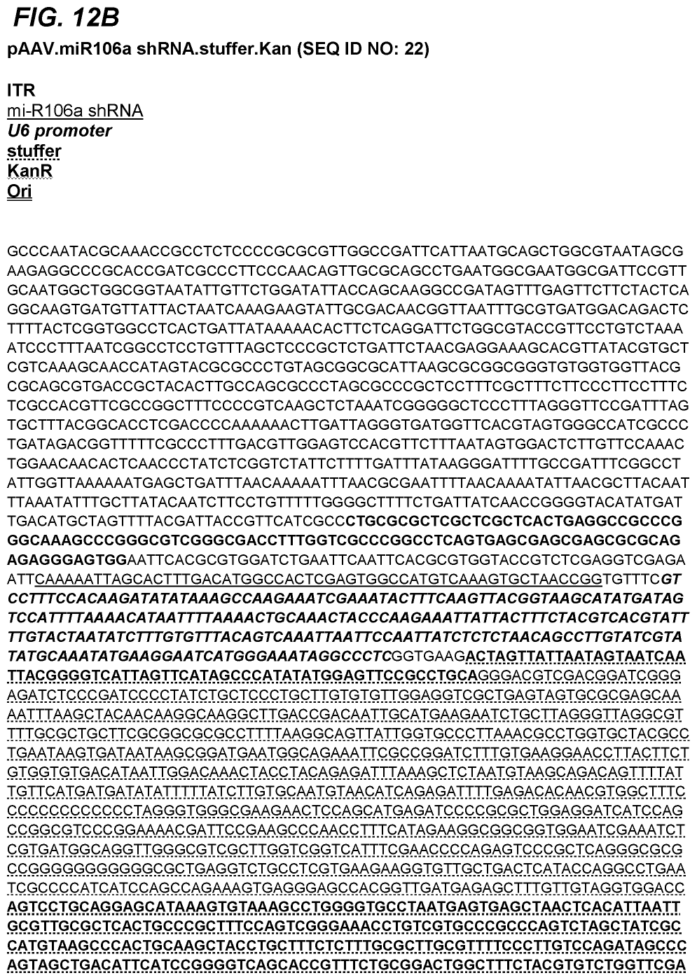

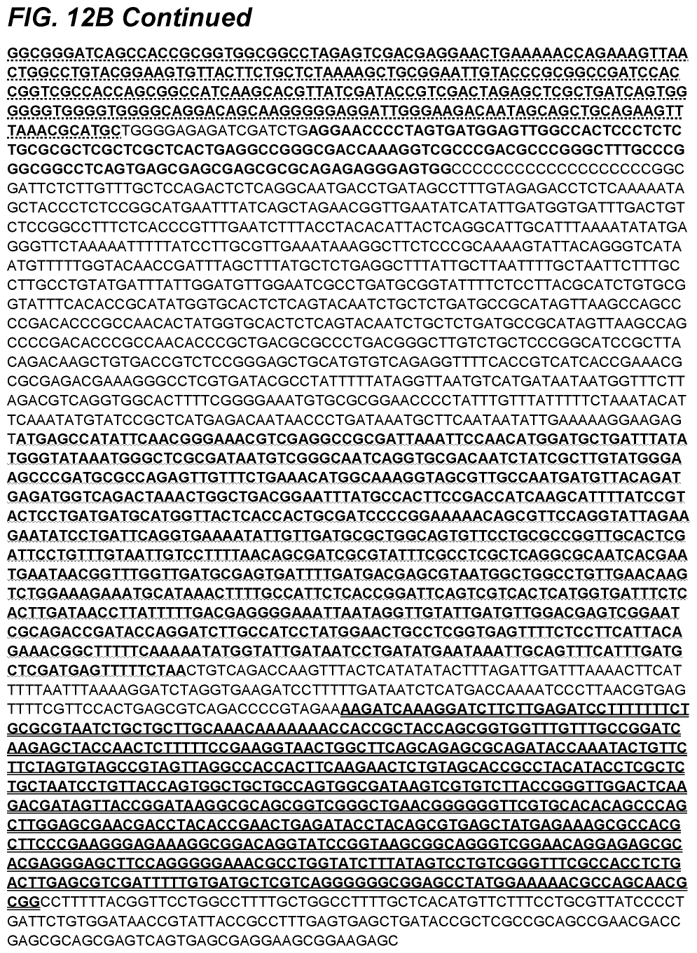

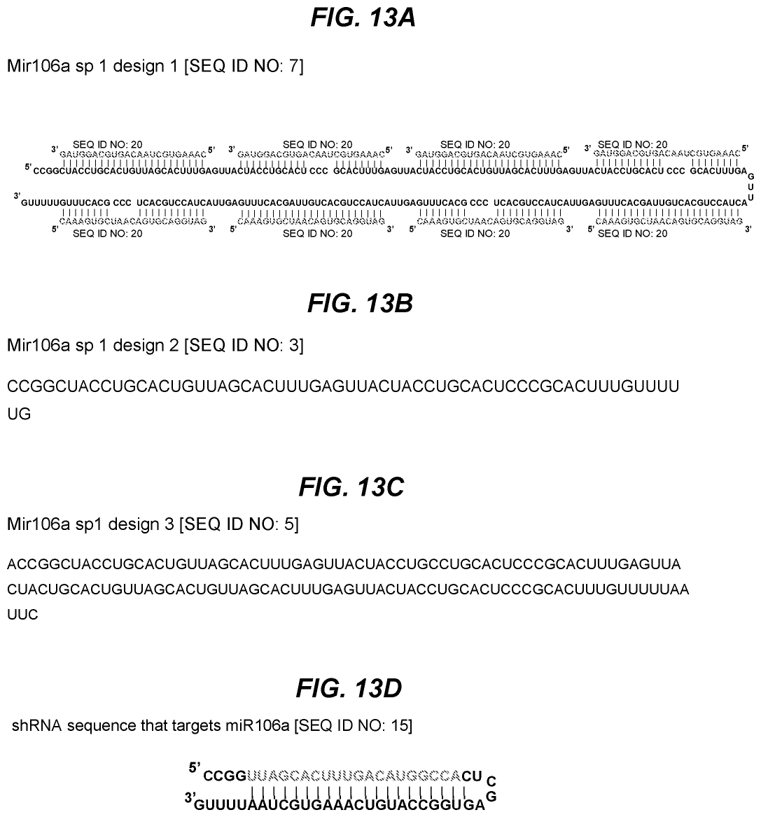

In some embodiments, the rAAV also may contain a stuffer sequence. The stuffer sequence is included in the vector to maintain optimal packaging length of the viral vector construct. The length of the stuffer sequence depends on the length of the sponge cassette. For example, the vectors contain a stuffer sequence that ranges in length from 1000 to 1500 base in length, or ranges from 500 to 2000 bases in length or ranges in 100 to 1000 bases in length. Exemplary stuffer sequences are 100 bases in length, or 200 bases in length, or 300 bases in length, or 400 bases in length, or 500 bases in length, or 600 bases in length, or 700 bases in length, or 800 bases in length, or 900 bases in length, or 1000 bases in length, or 1100 bases in length, or 1200 bases in length, or 1300 bases in length, or 1400 bases in length, or 1500 bases in length, or 1600 bases in length, or 1700 bases in length, or 1800 bases in length, or 1900 bases in length, or 2000 bases in length. To date, none of the FDA approved stuffer sequences are readily available. There are, however, several plasmid backbones that are approved by FDA for the human administration. Small DNA fragments were picked from these plasmids which do not correspond to any essential DNA sequences necessary for selection and replication of the plasmid or the elements of the transcriptional units. Exemplary plasmid backbones are listed in Table 2 and shown in FIGS. 12A-12B. The DNA elements from different plasmids were arranged in tandem to generate a complete, 1350 bp stuffer sequence (SEQ ID NO: 11).

Thus, in some aspects, the disclosure uses U6 shRNA molecules to further inhibit, knockdown, or interfere with miRNA of interest expression associated with X-linked disorders. Traditional small/short hairpin RNA (shRNA) sequences are usually transcribed inside the cell nucleus from a vector containing a Pol III promoter such as U6. The endogenous U6 promoter normally controls expression of the U6 RNA, a small RNA involved in splicing, and has been well-characterized [Kunkel et al., Nature. 322(6074):73-7 (1986); Kunkel et al., Genes Dev. 2(2):196-204 (1988); Paule et al., Nucleic Acids Res. 28(6):1283-98 (2000)]. In some aspects, the U6 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002)] because (1) the promoter is recognized by RNA polymerase III (poly III) and controls high-level, constitutive expression of shRNA; and (2) the promoter is active in most mammalian cell types. In some aspects, the promoter is a type III Pol III promoter in that all elements required to control expression of the shRNA are located upstream of the transcription start site (Paule et al., Nucleic Acids Res. 28(6):1283-98 (2000)). The disclosure includes both murine and human U6 or H1 promoters. In some embodiments, the U6 promoter is in the reverse orientation and it is on the complentary strand of the AAV genome. The shRNA containing the sense and antisense sequences from a target gene connected by a loop is transported from the nucleus into the cytoplasm where Dicer processes it into small/short interfering RNAs (siRNAs). In any of these embodiments, the shRNA is in the reverse orientation and therefore the shRNA is on complementary strand of the AAV genome.

As an understanding of natural RNAi pathways has developed, researchers have designed artificial shRNAs for use in regulating expression of target genes for treating disease. Several classes of small RNAs are known to trigger RNAi processes in mammalian cells, including short (or small) interfering RNA (siRNA), and short (or small) hairpin RNA (shRNA) and microRNA (miRNA), which constitute a similar class of vector-expressed triggers [Davidson et al., Nat. Rev. Genet. 12:329-40, 2011; Harper, Arch. Neurol. 66:933-8, 2009]. ShRNAs and miRNAs are expressed in vivo from plasmid- or virus-based vectors and may thus achieve long term gene silencing with a single administration, for as long as the vector is present within target cell nuclei and the driving promoter is active (Davidson et al., Methods Enzymol. 392:145-73, 2005). Importantly, this vector-expressed approach leverages the decades-long advancements already made in the muscle gene therapy field, but instead of expressing protein coding genes, the vector cargo in RNAi therapy strategies are artificial shRNA or miRNA cassettes targeting disease genes-of-interest. This strategy is used to express a natural miRNA. Each shRNA/miRNA is based on hsa-miR-30a sequences and structure. The natural mir-30a mature sequences are replaced by unique sense and antisense sequences derived from the target miRNA.

### MiRNAs Inactivating the Genes on the X Chromosome

In many X-linked disorder, during development, each cell randomly inactivates one of the two X chromosomes in females. Thus, females contain a mix of cells expressing either a healthy copy of the X-linked gene or the mutated copy, depending on which X chromosome they inactivated. The gene therapy approach disclosed herein takes advantage of the fact that each cell that expresses the mutated form of the X-linked gene, also contains the natural backup copy of the X-linked gene on the inactivated X chromosome. Thus, reactivation of parts of the silenced chromosome allow re-expression of the healthy gene.

As disclosed herein, a CRISPR/Cas9-based screen was carried to identify small non-coding RNAs involved in silencing of inactive X chromosome (Xi). Certain genes associated with X-linked disorders (X-linked genes) are located on the X chromosome, their allele-specific expression pattern is determined by X chromosome inactivation (XCI), an epigenetic mechanism that randomly inactivates one of the female X chromosomes. Certain small non-coding RNAs such as miRNAs can be epigenetic regulators of XCI. Such miRNAs (e.g. miR106a) inhibit the expression and/or activity of genes associated with X-linked disorders (e.g. MECP2 gene). Inhibition of these X-linked miRNAs targets increases the expression and/or activity of genes associated with X-linked disorders.

In various embodiments, the disclosure provides for vectors comprising a sponge cassette that targets one or more miRNAs of interest. The disclosure also provides for vectors comprising small RNA that target one or more miRNAs of interest. Targeting the miRNA of interest, either by the sponge or the small RNA results in binding and inactivation of the miRNA of interest, inhibition of expression of the miRNA of interest, and/or increasing the expression and/or activity of genes associated with X-linked disorders.

### Methyl CpG Binding Protein 2

The Methyl CpG binding protein 2 (MECP2) gene codes for MECP2 protein. MECP2 is a nuclear protein that functions as an important epigenetic reader, and repressor of thousands of genes in the central nervous system with regional and cell type specific alterations in gene expression. In 95% of typical Rett syndrome (RTT) cases, the disease is caused by deficiency of MECP2, a key regulator of gene expression in the central nervous system (CNS). Underlying clinical phenotypes of RTT is a global neuronal phenotype featuring compaction of neurons characterized by smaller soma and shortened and fewer neurites. Furthermore, clinically, and animal modeling has shown a direct connection between disease severity and neuroanatomical changes dependent on various MECP2 mutations.

The feasibility and safety of expressing Xi-linked MECP2 in vivo was assessed using small molecule inhibitors of phosphoinositide-dependent protein kinase 1 and activin A receptor type 1 (2, 3). Expression of Xi-linked genes did not cause any adverse effects in the treated animals and no off-target effects in tissues, such as liver were observed (2).

In various embodiments, the disclosure provides vectors or compositions comprising a sponge cassette or small RNA targeting a miRNA of interest which regulates MECP2 gene expression. In various embodiments, the expression of the sponge cassette or small RNA activates expression of the MECP2 gene.

### Rett Syndrome and X-Linked Disorders

Any of the vectors disclosed herein may be used to treat X-linked disorders. For example, any of the vectors disclosed herein may be used to treat Rett syndrome. Rett syndrome (RTT) is a neurodevelopmental disorder that affects girls almost exclusively at an incidence of 1 in ˜10,000 live births.

X-linked disorders which may be treated with any of the disclosed vectors include, but are not limited to rett syndrome, hemophilia A, hemophilia B, Dent's disease 1, Dent's disease 2, Albinism-deafness syndrome, Aldrich syndrome, Alport syndrome, Anaemia (hereditary hypochromic), Anemia, (sideroblastic with ataxia), Cataract, Charcot-Marie-Tooth, Color blindness, Diabetes (insipidus, nephrogenic), Dyskeratosis congenita, Ectodermal dysplasia, Faciogenital dysplasia, Fabry disease, Glucose-6-phosphate dehydrogenase deficiency, Glycogen storage disease type VIII, Gonadal dysgenesis, Testicular feminization syndrome, Addison's disease with cerebral sclerosis, Adrenal hypoplasia, Granulomatous disease, siderius X-linked mental retardation syndrome, Agammaglobulinaemia Bruton type, Choroidoretinal degeneration, Choroidaemia, Albinism (ocular), fragile X syndrome, Epileptic encephalopathy (early infantile 2), Hydrocephalus (aqueduct stenosis), Hypophosphataemic rickets, Lesch-Nyhan syndrome (hypoxanthine-guanine-phosphoribosyl transferase deficiency), incontinentia pigmenti, Kallmann syndrome, paroxysmal nocturnal hemoglobinuria, Spinal muscular atrophy 2, Spastic paraplegia, Keratosis follicularis spinulosa, Lowe (oculocerebrorenal) syndrome, Menkes syndrome, Renpenning Syndrome, Mental retardation, Coffin-Lowry syndrome, Microphthalmia (Lenz syndrome), Muscular dystrophy (Becker, Duchenne and Emery-Dreifuss types), Myotubular myopathy, Night blindness, Norrie's disease (pseudoglioma), Nystagmus, Orofaciodigital syndrome, Ornithine transcarbamylase deficiency (type I hyperammonaemia), Phosphoglycerate kinase deficiency, Phosphoribosylpyrophosphate synthetase deficiency, Retinitis pigmentosa, Retinoschisis, Muscular atrophy/Dihydrotestosterone receptor deficiency, Spinal muscular atrophy, Spondyloepiphyseal dysplasia tarda, Thrombocytopenia, Thyroxine-binding globulin, McLeod syndrome.

Further X-linked disorders which may be treated using any of the vectors disclosed herein are listed in Germain, “Chapter 7: General aspects of X-linked diseases” in Fabry Disease: Perspectives from 5 Years of FOS. Mehta A, Beck M, Sunder-Plassmann Gc editors. (Oxford: Oxford PharmaGenesis; 2006); Diseases and Disorders, (Marshall Cavendish, 2007) which are incorporated by reference.

In various embodiments, the disclosure provides vectors or compositions comprising a rAAV comprising any of the sponge cassettes or small RNA targeting one or more miRNA of interest which regulate X-linked gene expression. In various embodiments, the expression of the disclosed sponges or small RNA activates expression of the X-linked gene.

### Cancer

Exemplary conditions or disorders that can be treated with any of the vectors disclosed herein include cancers. In various embodiments, the cancer includes, but is not limited to gastric cancer, bone cancer, lung cancer, hepatocellular cancer, pancreatic cancer, kidney cancer, fibrotic cancer, breast cancer, myeloma, squamous cell carcinoma, colorectal cancer and prostate cancer. In related aspects the cancer is metastatic. In a related aspect, the metastasis includes metastasis to the bone or skeletal tissues, liver, lung, kidney or pancreas. It is contemplated that the methods herein reduce tumor size or tumor burden in the subject, and/or reduce metastasis in the subject. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

### AAV

In some aspects, the disclosure provides an adeno-associated virus (AAV) comprising any one or more nucleotide provided in the disclosure. In various embodiments, the one or more nucleotides are a microRNA sponge as disclosed herein. In various embodiments, the gene therapy vector is a single-stranded or self-complementary adeno-associated viral vector serotype 9 (AAV9) or similar vectors, such as AAV8, AAV10, Anc80, and AAV rh74. Recombinant AAV genomes of the disclosure comprise one or more miRNA sponge molecule(s) and one or more AAV ITRs flanking a nucleotide molecule. AAV DNA in the rAAV genomes may be from any AAV serotype for which a recombinant virus can be derived including, but not limited to, AAV serotypes AAV-B1, AAVrh.74, AAV-1, AAV-2, AAV-3, AAV-4, AAV-5, AAV-6, AAV-7, AAV-8, AAV-9, AAV-10, AAV-11, AAV-12, AAV-13, Anc80, or AAV7m8 or their derivatives. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

The disclosure also provides any one or more of the nucleotide sequences of the disclosure and any one or more of the AAV of the disclosure in a composition. In some aspects, the composition also comprises a diluent, an excipient, and/or an acceptable carrier. In some aspects, the carrier is a pharmaceutically acceptable carrier or a physiologically acceptable carrier.

In various embodiments, the gene therapy vector contains microRNA sponge cassettes that competitively inhibit the mature miR106a. In related embodiments, the sponges are tandem multiplexes of either perfectly or imperfectly complementary sequences to the mature microRNA 106a. MicroRNA 106a was previously identified by to regulate X chromosome inactivation by interacting with the Xist non-coding RNA. The binding of the sponges to the microRNA will induce its degradation and therefore interferes with X chromosome silencing. In various embodiments, expression of the microRNA sponges will be controlled by the U6.

In various embodiments, the gene therapy vector may be delivered via one of the following injection methods or using a combination of several of the injection methods: intravenous delivery, delivery through the cerebrospinal fluid (CSF) via lumbar intrathecal injection or other injection methods accessing the CSF.

In various embodiments, CSF delivery in humans or large animal species, the viral vector may be mixed with a contrast agent (Omnipaque or similar). In related embodiments, the contrast agent compositions may comprise a non-ionic, low-osmolar contrast agent. In related embodiments, the compositions may comprise a non-ionic, low-osmolar contrast agent is selected from the group consisting of iobitridol, iohexol, iomeprol, iopamidol, iopentol, iopromide, ioversol, ioxilan, and combinations thereof. In certain embodiments, immediately after CSF injection, patients may be held in a Trendelenburg position with head tilted downwards in a 15-30 degree angle for 5, 10, or 15 minutes. In related embodiments, CSF doses will range between 1e13 viral genomes (vg) per patient −1e15 vg/patient based on age groups. In various embodiments, intravenous delivery doses will range between 1e1 3 vg/kilogram (kg) body weight and 2e14 vg/kg.

In various embodiments, the vector may be used for additional diseases caused by loss of function mutation on genes found on the X chromosome, such as other X-linked disorders, e.g. DDX3X syndrome and Fragile X syndrome.

Self-complementary AAV (scAAV) vectors are also contemplated for use in the present disclosure. ScAAV vectors are generated by reducing the vector size to approximately 2500 base pairs, which comprise 2200 base pairs of unique transgene sequence plus two copies of the 145 base pair ITR packaged as a dimer. The scAAV have the ability to re-fold into double stranded DNA templates for expression. McCarthy, Mol. Therap. 16(10): 1648-1656, 2008.

DNA plasmids of the disclosure comprise a rAAV genome. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell, are known in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the disclosure comprise a rAAV genome. Embodiments include, but are not limited to, the rAAV named “pAAV.miR106a Sponge.Stuffer.Kan” encoding the miR106a sponge, encoded by the nucleotide sequence set out in SEQ ID NO: 21. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the disclosure are set out in International Patent Application No. PCT/US2012/047999 (WO 2013/016352) incorporated by reference herein in its entirety.

The rAAV may be purified by methods such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising a disclosed rAAV. Compositions of the disclosure comprise rAAV in a pharmaceutically acceptable carrier. The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the disclosure will vary depending, for example, on the particular rAAV, the mode of administration, the treatment goal, the individual, and the cell type(s) being targeted, and may be determined by methods known in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg).

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with the disclosed methods is FSHD.

Combination therapies are also contemplated by the disclosure. Combination as used herein includes both simultaneous treatment and sequential treatments. Combinations of methods of the disclosure with standard medical treatments (e.g., corticosteroids) are specifically contemplated, as are combinations with novel therapies.

Administration of an effective dose of the compositions may be by routes known in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the miRNA sponge or miRNA small RNA.

The disclosure provides for local administration and systemic administration of an effective dose of recombinant AAV and compositions of the disclosure. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present disclosure may be accomplished by using any physical method that will transport the rAAV recombinant vector into the target tissue of an animal. Administration according to the disclosure includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosed methods and compositions. The rAAV can be used with any pharmaceutically acceptable carrier for ease of administration and handling.

Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the disclosure results in sustained expression of microRNA sponge cassettes. The present disclosure thus provides methods of administering/delivering rAAV which express microRNA sponges to an animal, preferably a human being. These methods include transducing tissues (including, but not limited to, tissues such as muscle, organs such as liver and brain, and glands such as salivary glands) with one or more disclosed rAAV. Transduction may be carried out with gene cassettes comprising tissue specific control elements.

The term “transduction” is used to refer to the administration/delivery of microRNA sponge cassettes to a recipient cell either in vivo or in vitro, via a replication-deficient rAAV resulting in expression of a microRNA sponge by the recipient cell.

The invention also provides for pharmaceutical compositions (or sometimes referred to herein as simply “compositions”) comprising any of the rAAV vectors of the invention.

### Methods of Treatment

The terms “treat,” “treated,” “treating,” “treatment,” and the like are meant to refer to reducing or ameliorating a disorder and/or symptoms associated therewith (e.g., Rett syndrome, other X-linked disorders or cancer). ‘Treating” may refer to administration of the combination therapy to a subject after the onset, or suspected onset, of a Rett syndrome, other X-linked disorders or cancer. “Treating” includes the concepts of “alleviating”, which refers to lessening the frequency of occurrence or recurrence, or the severity, of any symptoms or other ill effects related to a Rett syndrome or other X-linked disorder and/or the side effects associated with such a disorder. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

In various embodiments, the disclosure provides a method of treating Rett syndrome, an X-linked disorder, or cancer.

The disclosure provides methods of administering recombinant AAV vectors comprising a microRNA sponge cassettes an effective dose (or doses, administered essentially simultaneously or doses given at intervals) of rAAV that encode one or more microRNA sponge cassettes targeting miR106a to a patient in need thereof.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications, patents and patent applications cited in this specification are herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims.

Sequences

SEQ ID NO: 1. miR106a Sponge RNA sequence targeting miR106a
CUACCUGCACUGUUAGCACUUUG
SEQ ID NO: 2. miR106a Sponge DNA sequence targeting miR106a
CTACCTGCACTGTTAGCACTTTG
SEQ ID NO: 3. mir106a sp1 design 2 RNA
CCGGCUACCUGCACUGUUAGCACUUUGAGUUACUACCUGCACUCCCGCACUUUGUUUUUG
SEQ ID NO: 4. mir106a sp1 design 2 DNA
CCGGCTACCTGCACTGTTAGCACTTTGAGTTACTACCTGCACTCCCGCACTTTGTTTTTG
SEQ ID NO: 5. mir106a sp1 design 3 RNA
ACCGGCUACCUGCACUGUUAGCACUUUGAGUUACUACCUGCCUGCACUCCCGCACUUUGAGU
UACUACUGCACUGUUAGCACUGUUAGCACUUUGAGUUACUACCUGCACUCCCGCACUUUGUU
UUUAAUUC
SEQ ID NO: 6. mir106a sp1 design 3 DNA
ACCGGCTACCTGCACTGTTAGCACTTTGAGTTACTACCTGCCTGCACTCCCGCACTTTGAGTTAC
TACTGCACTGTTAGCACTGTTAGCACTTTGAGTTACTACCTGCACTCCCGCACTTTGTTTTTAATT
C
SEQ ID NO: 7. miR106a Sponge cassette RNA
CCGGCUACCUGCACUGUUAGCACUUUGAGUUACUACCUGCACUCCCGCACUUUGAGUUACUA
CCUGCACUGUUAGCACUUUGAGUUACUACCUGCACUCCCGCACUUUGAGUUACUACCUGCAC
UGUUAGCACUUUGAGUUACUACCUGCACUCCCGCACUUUGAGUUACUACCUGCACUGUUAGC
ACUUUGAGUUACUACCUGCACUCCCGCACUUUGUUUUUG
SEQ ID NO: 8. miR106a Sponge cassette DNA
CCGGCTACCTGCACTGTTAGCACTTTGAGTTACTACCTGCACTCCCGCACTTTGAGTTACTACCT
GCACTGTTAGCACTTTGAGTTACTACCTGCACTCCCGCACTTTGAGTTACTACCTGCACTGTTAG
CACTTTGAGTTACTACCTGCACTCCCGCACTTTGAGTTACTACCTGCACTGTTAGCACTTTGAGTT
ACTACCTGCACTCCCGCACTTTGTTTTTG
SEQ ID NO: 9. mITR
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGG
SEQ ID NO: 10. U6 promoter
GTCCTTTCCACAAGATATATAAAGCCAAGAAATCGAAATACTTTCAAGTTACGGTAAGCATATGAT
AGTCCATTTTAAAACATAATTTTAAAACTGCAAACTACCCAAGAAATTATTACTTTCTACGTCACGT
ATTTTGTACTAATATCTTTGTGTTTACAGTCAAATTAATTCCAATTATCTCTCTAACAGCCTTGTATC
GTATATGCAAATATGAAGGAATCATGGGAAATAGGCCCTC
SEQ ID NO: 11. Stuffer
ACTAGTTATTAATAGTAATCAATTACGGGGTCATTAGTTCATAGCCCATATATGGAGTTCCGCCTG
CAGGGACGTCGACGGATCGGGAGATCTCCCGATCCCCTATCTGCTCCCTGCTTGTGTGTTGGAG
GTCGCTGAGTAGTGCGCGAGCAAAATTTAAGCTACAACAAGGCAAGGCTTGACCGACAATTGCA
TGAAGAATCTGCTTAGGGTTAGGCGTTTTGCGCTGCTTCGCGGCGCGCCTTTTAAGGCAGTTATT
GGTGCCCTTAAACGCCTGGTGCTACGCCTGAATAAGTGATAATAAGCGGATGAATGGCAGAAAT
TCGCCGGATCTTTGTGAAGGAACCTTACTTCTGTGGTGTGACATAATTGGACAAACTACCTACAG
AGATTTAAAGCTCTAATGTAAGCAGACAGTTTTATTGTTCATGATGATATATTTTTATCTTGTGCAA
TGTAACATCAGAGATTTTGAGACACAACGTGGCTTTCCCCCCCCCCCCCTAGGGTGGGCGAAGA
ACTCCAGCATGAGATCCCCGCGCTGGAGGATCATCCAGCCGGCGTCCCGGAAAACGATTCCGA
AGCCCAACCTTTCATAGAAGGCGGCGGTGGAATCGAAATCTCGTGATGGCAGGTTGGGCGTCG
CTTGGTCGGTCATTTCGAACCCCAGAGTCCCGCTCAGGGCGCGCCGGGGGGGGGGGCGCTGA
GGTCTGCCTCGTGAAGAAGGTGTTGCTGACTCATACCAGGCCTGAATCGCCCCATCATCCAGCC
AGAAAGTGAGGGAGCCACGGTTGATGAGAGCTTTGTTGTAGGTGGACCAGTCCTGCAGGAGCAT
AAAGTGTAAAGCCTGGGGTGCCTAATGAGTGAGCTAACTCACATTAATTGCGTTGCGCTCACTGC
CCGCTTTCCAGTCGGGAAACCTGTCGTGCCCGCCCAGTCTAGCTATCGCCATGTAAGCCCACTG
CAAGCTACCTGCTTTCTCTTTGCGCTTGCGTTTTCCCTTGTCCAGATAGCCCAGTAGCTGACATT
CATCCGGGGTCAGCACCGTTTCTGCGGACTGGCTTTCTACGTGTCTGGTTCGAGGCGGGATCAG
CCACCGCGGTGGCGGCCTAGAGTCGACGAGGAACTGAAAAACCAGAAAGTTAACTGGCCTGTA
CGGAAGTGTTACTTCTGCTCTAAAAGCTGCGGAATTGTACCCGCGGCCGATCCACCGGTCGCCA
CCAGCGGCCATCAAGCACGTTATCGATACCGTCGACTAGAGCTCGCTGATCAGTGGGGGGTGG
GGTGGGGCAGGACAGCAAGGGGGAGGATTGGGAAGACAATAGCAGCTGCAGAAGTTTAAACGC
ATGC
SEQ ID NO: 12. ITR
AGGAACCCCTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCGAGCGAGCG
CGCAGAGAGGGAGTGG
SEQ ID NO: 14. mITR
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGG
SEQ ID NO: 18 Spacer 1
AGTTA
SEQ ID NO: 19 Spacer 2
AGUUA
SEQ ID NO: 20 Mouse miR106a-5p sequence
CAAAGUGCUAACAGUGCAGGUAG
SEQ ID NO: 21. pAAV.miR106a Sponge.Stuffer.Kan
GCCCAATACGCAAACCGCCTCTCCCCGCGCGTTGGCCGATTCATTAATGCAGCTGGCGTAATAG
CGAAGAGGCCCGCACCGATCGCCCTTCCCAACAGTTGCGCAGCCTGAATGGCGAATGGCGATT
CCGTTGCAATGGCTGGCGGTAATATTGTTCTGGATATTACCAGCAAGGCCGATAGTTTGAGTTCT
TCTACTCAGGCAAGTGATGTTATTACTAATCAAAGAAGTATTGCGACAACGGTTAATTTGCGTGAT
GGACAGACTCTTTTACTCGGTGGCCTCACTGATTATAAAAACACTTCTCAGGATTCTGGCGTACC
GTTCCTGTCTAAAATCCCTTTAATCGGCCTCCTGTTTAGCTCCCGCTCTGATTCTAACGAGGAAA
GCACGTTATACGTGCTCGTCAAAGCAACCATAGTACGCGCCCTGTAGCGGCGCATTAAGCGCGG
CGGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGGGGG
CTCCCTTTAGGGTTCCGATTTAGTGCTTTACGGCACCTCGACCCCAAAAAACTTGATTAGGGTGA
TGGTTCACGTAGTGGGCCATCGCCCTGATAGACGGTTTTTCGCCCTTTGACGTTGGAGTCCACG
TTCTTTAATAGTGGACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGATTTAACAAAAATTT
AACGCGAATTTTAACAAAATATTAACGCTTACAATTTAAATATTTGCTTATACAATCTTCCTGTTTTT
GGGGCTTTTCTGATTATCAACCGGGGTACATATGATTGACATGCTAGTTTTACGATTACCGTTCAT
CGCCCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGAATTCACGCGTGGA
TCTGAATTCAATTCACGCGTGGTACCGTCTCGAGGTCGAGAATTCAAAAACAAAGTGCGGGAGT
GCAGGTAGTAACTCAAAGTGCTAACAGTGCAGGTAGTAACTCAAAGTGCGGGAGTGCAGGTAGT
AACTCAAAGTGCTAACAGTGCAGGTAGTAACTCAAAGTGCGGGAGTGCAGGTAGTAACTCAAAG
TGCTAACAGTGCAGGTAGTAACTCAAAGTGCGGGAGTGCAGGTAGTAACTCAAAGTGCTAACAG
TGCAGGTAGCCGGTGTTTCGTCCTTTCCACAAGATATATAAAGCCAAGAAATCGAAATACTTTCAA
GTTACGGTAAGCATATGATAGTCCATTTTAAAACATAATTTTAAAACTGCAAACTACCCAAGAAATT
ATTACTTTCTACGTCACGTATTTTGTACTAATATCTTTGTGTTTACAGTCAAATTAATTCCAATTATC
TCTCTAACAGCCTTGTATCGTATATGCAAATATGAAGGAATCATGGGAAATAGGCCCTCGGTGAA
GACTAGTTATTAATAGTAATCAATTACGGGGTCATTAGTTCATAGCCCATATATGGAGTTCCGCCT
GCAGGGACGTCGACGGATCGGGAGATCTCCCGATCCCCTATCTGCTCCCTGCTTGTGTGTTGGA
GGTCGCTGAGTAGTGCGCGAGCAAAATTTAAGCTACAACAAGGCAAGGCTTGACCGACAATTGC
ATGAAGAATCTGCTTAGGGTTAGGCGTTTTGCGCTGCTTCGCGGCGCGCCTTTTAAGGCAGTTAT
TGGTGCCCTTAAACGCCTGGTGCTACGCCTGAATAAGTGATAATAAGCGGATGAATGGCAGAAA
TTCGCCGGATCTTTGTGAAGGAACCTTACTTCTGTGGTGTGACATAATTGGACAAACTACCTACA
GAGATTTAAAGCTCTAATGTAAGCAGACAGTTTTATTGTTCATGATGATATATTTTTATCTTGTGCA
ATGTAACATCAGAGATTTTGAGACACAACGTGGCTTTCCCCCCCCCCCCCTAGGGTGGGCGAAG
AACTCCAGCATGAGATCCCCGCGCTGGAGGATCATCCAGCCGGCGTCCCGGAAAACGATTCCG
AAGCCCAACCTTTCATAGAAGGCGGCGGTGGAATCGAAATCTCGTGATGGCAGGTTGGGCGTC
GCTTGGTCGGTCATTTCGAACCCCAGAGTCCCGCTCAGGGCGCGCCGGGGGGGGGGGCGCTG
AGGTCTGCCTCGTGAAGAAGGTGTTGCTGACTCATACCAGGCCTGAATCGCCCCATCATCCAGC
CAGAAAGTGAGGGAGCCACGGTTGATGAGAGCTTTGTTGTAGGTGGACCAGTCCTGCAGGAGC
ATAAAGTGTAAAGCCTGGGGTGCCTAATGAGTGAGCTAACTCACATTAATTGCGTTGCGCTCACT
GCCCGCTTTCCAGTCGGGAAACCTGTCGTGCCCGCCCAGTCTAGCTATCGCCATGTAAGCCCAC
TGCAAGCTACCTGCTTTCTCTTTGCGCTTGCGTTTTCCCTTGTCCAGATAGCCCAGTAGCTGACA
TTCATCCGGGGTCAGCACCGTTTCTGCGGACTGGCTTTCTACGTGTCTGGTTCGAGGCGGGATC
AGCCACCGCGGTGGCGGCCTAGAGTCGACGAGGAACTGAAAAACCAGAAAGTTAACTGGCCTG
TACGGAAGTGTTACTTCTGCTCTAAAAGCTGCGGAATTGTACCCGCGGCCGATCCACCGGTCGC
CACCAGCGGCCATCAAGCACGTTATCGATACCGTCGACTAGAGCTCGCTGATCAGTGGGGGGT
GGGGTGGGGCAGGACAGCAAGGGGGAGGATTGGGAAGACAATAGCAGCTGCAGAAGTTTAAAC
GCATGCTGGGGAGAGATCGATCTGAGGAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCGG
GCGGCCTCAGTGAGCGAGCGAGCGCGCAGAGAGGGAGTGGCCCCCCCCCCCCCCCCCCCGG
CGATTCTCTTGTTTGCTCCAGACTCTCAGGCAATGACCTGATAGCCTTTGTAGAGACCTCTCAAA
AATAGCTACCCTCTCCGGCATGAATTTATCAGCTAGAACGGTTGAATATCATATTGATGGTGATTT
GACTGTCTCCGGCCTTTCTCACCCGTTTGAATCTTTACCTACACATTACTCAGGCATTGCATTTAA
AATATATGAGGGTTCTAAAAATTTTTATCCTTGCGTTGAAATAAAGGCTTCTCCCGCAAAAGTATT
ACAGGGTCATAATGTTTTTGGTACAACCGATTTAGCTTTATGCTCTGAGGCTTTATTGCTTAATTTT
GCTAATTCTTTGCCTTGCCTGTATGATTTATTGGATGTTGGAATCGCCTGATGCGGTATTTTCTCC
TTACGCATCTGTGCGGTATTTCACACCGCATATGGTGCACTCTCAGTACAATCTGCTCTGATGCC
GCATAGTTAAGCCAGCCCCGACACCCGCCAACACTATGGTGCACTCTCAGTACAATCTGCTCTG
ATGCCGCATAGTTAAGCCAGCCCCGACACCCGCCAACACCCGCTGACGCGCCCTGACGGGCTT
GTCTGCTCCCGGCATCCGCTTACAGACAAGCTGTGACCGTCTCCGGGAGCTGCATGTGTCAGAG
GTTTTCACCGTCATCACCGAAACGCGCGAGACGAAAGGGCCTCGTGATACGCCTATTTTTATAG
GTTAATGTCATGATAATAATGGTTTCTTAGACGTCAGGTGGCACTTTTCGGGGAAATGTGCGCGG
AACCCCTATTTGTTTATTTTTCTAAATACATTCAAATATGTATCCGCTCATGAGACAATAACCCTGA
TAAATGCTTCAATAATATTGAAAAAGGAAGAGTATGAGCCATATTCAACGGGAAACGTCGAGGCC
GCGATTAAATTCCAACATGGATGCTGATTTATATGGGTATAAATGGGCTCGCGATAATGTCGGGC
AATCAGGTGCGACAATCTATCGCTTGTATGGGAAGCCCGATGCGCCAGAGTTGTTTCTGAAACAT
GGCAAAGGTAGCGTTGCCAATGATGTTACAGATGAGATGGTCAGACTAAACTGGCTGACGGAAT
TTATGCCACTTCCGACCATCAAGCATTTTATCOGTACTCCTGATGATGCATGGTTACTCACCACTG
CGATCCCCGGAAAAACAGCGTTCCAGGTATTAGAAGAATATCCTGATTCAGGTGAAAATATTGTT
GATGCGCTGGCAGTGTTCCTGCGCCGGTTGCACTCGATTCCTGTTTGTAATTGTCCTTTTAACAG
CGATCGCGTATTTCGCCTCGCTCAGGCGCAATCACGAATGAATAACGGTTTGGTTGATGCGAGT
GATTTTGATGACGAGCGTAATGGCTGGCCTGTTGAACAAGTCTGGAAAGAAATGCATAAACTTTT
GCCATTCTCACCGGATTCAGTCGTCACTCATGGTGATTTCTCACTTGATAACCTTATTTTTGACGA
GGGGAAATTAATAGGTTGTATTGATGTTGGACGAGTCGGAATCGCAGACCGATACCAGGATCTT
GCCATCCTATGGAACTGCCTCGGTGAGTTTTCTCCTTCATTACAGAAACGGCTTTTTCAAAAATAT
GGTATTGATAATCCTGATATGAATAAATTGCAGTTTCATTTGATGCTCGATGAGTTTTTCTAACTGT
CAGACCAAGTTTACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTTCCACTGAGCG
TCAGACCCCGTAGAAAAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTTCTTCTAGTGTAGCCG
TAGTTAGGCCACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGACGATAGTTA
CCGGATAAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGCTTCCCGAA
GGGAGAAAGGCGGACAGGTATCCGGTAAGCGGCAGGGTCGGAACAGGAGAGCGCACGAGGGA
GCTTCCAGGGGGAAACGCCTGGTATCTTTATAGTCCTGTCGGGTTTCGCCACCTCTGACTTGAG
CGTCGATTTTTGTGATGCTCGTCAGGGGGGCGGAGCCTATGGAAAAACGCCAGCAACGCGGCC
TTTTTACGGTTCCTGGCCTTTTGCTGGCCTTTTGCTCACATGTTCTTTCCTGCGTTATCCCCTGAT
TCTGTGGATAACCGTATTACCGCCTTTGAGTGAGCTGATACCGCTCGCCGCAGCCGAACGACCG
AGCGCAGCGAGTCAGTGAGCGAGGAAGCGGAAGAGC
SEQ ID NO: 22. pAAV.miR1 06a shRNA.stuffer.Kan
GCCCAATACGCAAACCGCCTCTCCCCGCGCGTTGGCCGATTCATTAATGCAGCTGGCGTAATAG
CGAAGAGGCCCGCACCGATCGCCCTTCCCAACAGTTGCGCAGCCTGAATGGCGAATGGCGATT
CCGTTGCAATGGCTGGCGGTAATATTGTTCTGGATATTACCAGCAAGGCCGATAGTTTGAGTTCT
TCTACTCAGGCAAGTGATGTTATTACTAATCAAAGAAGTATTGCGACAACGGTTAATTTGCGTGAT
GGACAGACTCTTTTACTCGGTGGCCTCACTGATTATAAAAACACTTCTCAGGATTCTGGCGTACC
GTTCCTGTCTAAAATCCCTTTAATCGGCCTCCTGTTTAGCTCCCGCTCTGATTCTAACGAGGAAA
GCACGTTATACGTGCTCGTCAAAGCAACCATAGTACGCGCCCTGTAGCGGCGCATTAAGCGCGG
CGGGTGTGGTGGTTACGCGCAGCGTGACCGCTACACTTGCCAGCGCCCTAGCGCCCGCTCCTT
TCGCTTTCTTCCCTTCCTTTCTCGCCACGTTCGCCGGCTTTCCCCGTCAAGCTCTAAATCGGGGG
CTCCCTTTAGGGTTCCGATTTAGTGCTTTACGGCACCTCGACCCCAAAAAACTTGATTAGGGTGA
TGGTTCACGTAGTGGGCCATCGCCCTGATAGACGGTTTTTCGCCCTTTGACGTTGGAGTCCACG
TTCTTTAATAGTGGACTCTTGTTCCAAACTGGAACAACACTCAACCCTATCTCGGTCTATTCTTTT
GATTTATAAGGGATTTTGCCGATTTCGGCCTATTGGTTAAAAAATGAGCTGATTTAACAAAAATTT
AACGCGAATTTTAACAAAATATTAACGCTTACAATTTAAATATTTGCTTATACAATCTTCCTGTTTTT
GGGGCTTTTCTGATTATCAACCGGGGTACATATGATTGACATGCTAGTTTTACGATTACCGTTGAT
CGCCCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGAATTCACGCGTGGA
TCTGAATTCAATTCACGCGTGGTACCGTCTCGAGGTCGAGAATTCAAAAATTAGCACTTTGACAT
GGCCACTCGAGTGGCCATGTCAAAGTGCTAACCGGTGTTTCGTCCTTTCCACAAGATATATAAAG
CCAAGAAATCGAAATACTTTCAAGTTACGGTAAGCATATGATAGTCCATTTTAAAACATAATTTTAA
AACTGCAAACTACCCAAGAAATTATTACTTTCTACGTCACGTATTTTGTACTAATATCTTTGTGTTT
ACAGTCAAATTAATTCCAATTATCTCTCTAACAGCCTTGTATCGTATATGCAAATATGAAGGAATCA
TGGGAAATAGGCCCTCGGTGAAGACTAGTTATTAATAGTAATCAATTACGGGGTCATTAGTTCAT
AGCCCATATATGGAGTTCCGCCTGCAGGGACGTCGACGGATCGGGAGATCTCCCGATCCCCTAT
CTGCTCCCTGCTTGTGTGTTGGAGGTCGCTGAGTAGTGCGCGAGCAAAATTTAAGCTACAACAA
GGCAAGGCTTGACCGACAATTGCATGAAGAATCTGCTTAGGGTTAGGCGTTTTGCGCTGCTTCG
CGGCGCGCCTTTTAAGGCAGTTATTGGTGCCCTTAAACGCCTGGTGCTACGCCTGAATAAGTGA
TAATAAGCGGATGAATGGCAGAAATTCGCCGGATCTTTGTGAAGGAACCTTACTTCTGTGGTGTG
ACATAATTGGACAAACTACCTACAGAGATTTAAAGCTCTAATGTAAGCAGACAGTTTTATTGTTCA
TGATGATATATTTTTATCTTGTGCAATGTAACATCAGAGATTTTGAGACACAACGTGGCTTTCCCC
CCCCCCCCCTAGGGTGGGCGAAGAACTCCAGCATGAGATCCCCGCGCTGGAGGATCATCCAGC
CGGCGTCCCGGAAAACGATTCCGAAGCCCAACCTTTCATAGAAGGCGGCGGTGGAATCGAAATC
TCGTGATGGCAGGTTGGGCGTCGCTTGGTCGGTCATTTCGAACCCCAGAGTCCCGCTCAGGGC
GCGCCGGGGGGGGGGGCGCTGAGGTCTGCCTCGTGAAGAAGGTGTTGCTGACTCATACCAGG
CCTGAATCGCCCCATCATCCAGCCAGAAAGTGAGGGAGCCACGGTTGATGAGAGCTTTGTTGTA
GGTGGACCAGTCCTGCAGGAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCCGCCCAGTC
TAGCTATCGCCATGTAAGCCCACTGCAAGCTACCTGCTTTCTCTTTGCGCTTGCGTTTTCCCTTG
TCCAGATAGCCCAGTAGCTGACATTCATCCGGGGTCAGCACCGTTTCTGCGGACTGGCTTTCTA
CGTGTCTGGTTCGAGGCGGGATCAGCCACCGCGGTGGCGGCCTAGAGTCGACGAGGAACTGAA
AAACCAGAAAGTTAACTGGCCTGTACGGAAGTGTTACTTCTGCTCTAAAAGCTGCGGAATTGTAC
CCGCGGCCGATCCACCGGTCGCCACCAGCGGCCATCAAGCACGTTATCGATACCGTCGACTAG
AGCTCGCTGATCAGTGGGGGGTGGGGTGGGGCAGGACAGCAAGGGGGAGGATTGGGAAGACA
ATAGCAGCTGCAGAAGTTTAAACGCATGCTGGGGAGAGATCGATCTGAGGAACCCCTAGTGATG
GAGTTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAGGGAGTGG
CCCCCCCCCCCCCCCCCCCGGCGATTCTCTTGTTTGCTCCAGACTCTCAGGCAATGACCTGATA
GCCTTTGTAGAGACCTCTCAAAAATAGCTACCCTCTCCGGCATGAATTTATCAGCTAGAACGGTT
GAATATCATATTGATGGTGATTTGACTGTCTCCGGCCTTTCTCACCCGTTTGAATCTTTACCTACA
CATTACTCAGGCATTGCATTTAAAATATATGAGGGTTCTAAAAATTTTTATCCTTGCGTTGAAATAA
AGGCTTCTCCCGCAAAAGTATTACAGGGTCATAATGTTTTTGGTACAACCGATTTAGCTTTATGCT
CTGAGGCTTTATTGCTTAATTTTGCTAATTCTTTGCCTTGCCTGTATGATTTATTGGATGTTGGAAT
CGCCTGATGCGGTATTTTCTCCTTACGCATCTGTGCGGTATTTCACACCGCATATGGTGCACTCT
CAGTACAATCTGCTCTGATGCCGCATAGTTAAGCCAGCCCCGACACCCGCCAACACTATGGTGC
ACTCTCAGTACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGACGAAAGGGCCT
CGTGATACGCCTATTTTTATAGGTTAATGTCATGATAATAATGGTTTCTTAGACGTCAGGTGGCAC
TTTTCGGGGAAATGTGCGCGGAACCCCTATTTGTTTATTTTTCTAAATACATTCAAATATGTATCC
GCTCATGAGACAATAACCCTGATAAATGCTTCAATAATATTGAAAAAGGAAGAGTATGAGCCATAT
TCAACGGGAAACGTCGAGGCCGCGATTAAATTCCAACATGGATGCTGATTTATATGGGTATAAAT
GGGCTCGCGATAATGTCGGGCAATCAGGTGCGACAATCTATCGCTTGTATGGGAAGCCCGATGC
GCCAGAGTTGTTTCTGAAACATGGCAAAGGTAGCGTTGCCAATGATGTTACAGATGAGATGGTCA
GACTAAACTGGCTGACGGAATTTATGCCACTTCCGACCATCAAGCATTTTATCCGTACTCCTGAT
GATGCATGGTTACTCACCACTGCGATCCCCGGAAAAACAGCGTTCCAGGTATTAGAAGAATATCC
TGATTCAGGTGAAAATATTGTTGATGCGCTGGCAGTGTTCCTGCGCCGGTTGCACTCGATTCCTG
TTTGTAATTGTCCTTTTAACAGCGATCGCGTATTTCGCCTCGCTCAGGCGCAATCACGAATGAAT
AACGGTTTGGTTGATGCGAGTGATTTTGATGACGAGCGTAATGGCTGGCCTGTTGAACAAGTCT
GGAAAGAAATGCATAAACTTTTGCCATTCTCACCGGATTCAGTCGTCACTCATGGTGATTTCTCAC
TTGATAACCTTATTTTTGACGAGGGGAAATTAATAGGTTGTATTGATGTTGGACGAGTCGGAATC
GCAGACCGATACCAGGATCTTGCCATCCTATGGAACTGCCTCGGTGAGTTTTCTCCTTCATTACA
GAAACGGCTTTTTCAAAAATATGGTATTGATAATCCTGATATGAATAAATTGCAGTTTCATTTGATG
CTCGATGAGTTTTTCTAACTGTCAGACCAAGTTTACTCATATATACTTTAGATTGATTTAAAACTTC
ATTTTTAATTTAAAAGGATCTAGGTGAAGATCCTTTTTGATAATCTCATGACCAAAATCCCTTAACG
TGAGTTTTCGTTCCACTGAGCGTCAGACCCCGTAGAAAAGATCAAAGGATCTTCTTGAGATCCTT
TTTTTCTGCGCGTAATCTGCTGCTTGCAAACAAAAAAACCACCGCTACCAGCGGTGGTTTGTTTG
CGGATCAAGAGCTACCAACTCTTTTTCCGAAGGTAACTGGCTTCAGCAGAGCGCAGATACCAA
ATACTGTTCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACCG
GGTTGGACTCAAGACGATAGTTACCGGATAAGGCGCAGCGGTCGGGCTGAACGGGGGGTTCGT
GCACACAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGGGTCG
GAACAGGAGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTCACATGT
TCTTTCCTGCGTTATCCCCTGATTCTGTGGATAACCGTATTACCGCCTTTGAGTGAGCTGATACC
GCTCGCCGCAGCCGAACGACCGAGCGCAGCGAGTCAGTGAGCGAGGAAGCGGAAGAGC
SEQ ID NO: 23. miR106a shRNA DNA (shRNA target sequence bolded nucleotides 5-24)
CCGG [symbol] CTCGAGTGGCCATGTCAAAGTGCTAATTTTTG
SEQ ID NO: 24. miR106a shRNA RNA( shRNA target sequence bolded nucleotides 5-24)
CCGG [symbol] CUCGAGUGGCCAUGUCAAAGUGCUAAUUUUG
SEQ ID NO: 25. Human mir106a-5p
AAAAGUGCUUACAGUGCAGGUAG

Additional aspects and details of the disclosure will be apparent from the following examples, which are intended to be illustrative rather than limiting.

### Example 1

### A CRISPR/Cas9 Screen Identifies miR106a as an Epigenetic Regulator of XCI

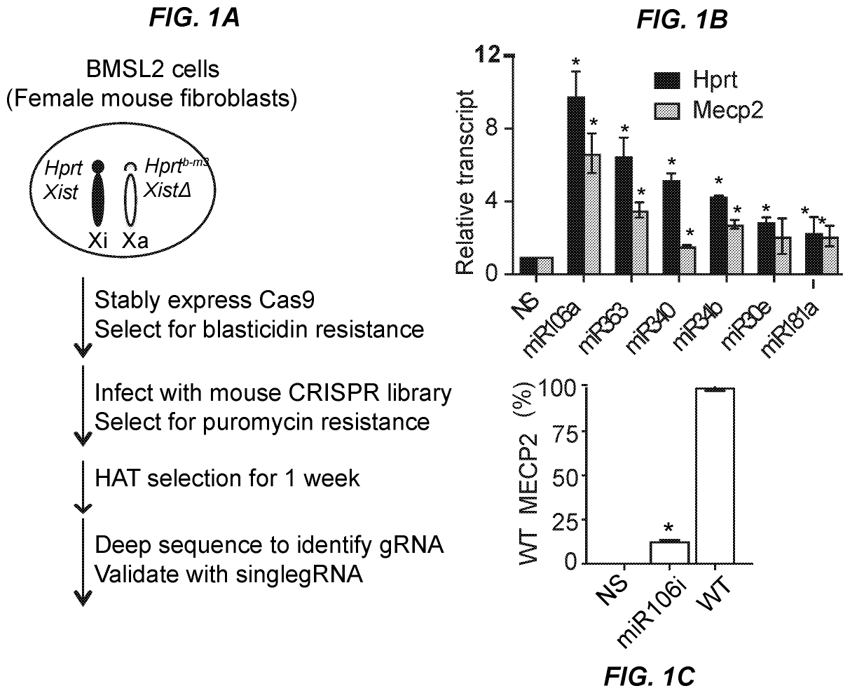

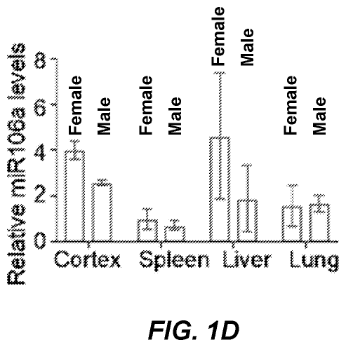

MiRNAs as epigenetic regulators of X chromosome inactivation (XCI) were identified through an unbiased CRISPR/Cas9 screen (FIG. 1A). A female mouse fibroblast reporter cell line (BMSL2) that bears a deletion in Xist promoter and Hprt gene enabled specific monitoring of Xi-linked Hprt, indicating X-reactivation (5, 6). To initiate the screen, a BMSL2 cell line stably expressing wild type Cas9 endonuclease was generated. After viral delivery of single guide RNAs (sgRNA) library, cells expressing Hprt from Xi were enriched in Hypoxanthine-aminopterin-thymidine selection media (for example, see (6)). Using next generation sequencing, six miRNAs were identified as XCI regulators that include, miR106a, miR363, miR181a, miR340, miR34b, and miR30e (data not shown). Along with miRNA, nineteen protein-coding XCIFs were also identified, including two factors that were previously identified through shRNA screen, ACVR1 and STC1 (6), thereby validating the screen.

The miRNAs were rank-ordered based on the reactivation of Hprt and MECP2 obtained with sgRNA-directed against the same target in multiple cellular models (FIG. 1B). The focus became the highest scoring candidate, miR106a (FIG. 1B), encoded by miRNA cluster on the X chromosome and highly expressed in mouse brain cortex (FIG. 1D). Moreover, the analysis of previously published miR106a-crosslinking immunoprecipitation data revealed multiple miR106a seed sequence in 5′ region of Xist RNA, a key regulator of XCI (data not shown), supporting the proposed miR106a function in XCI.

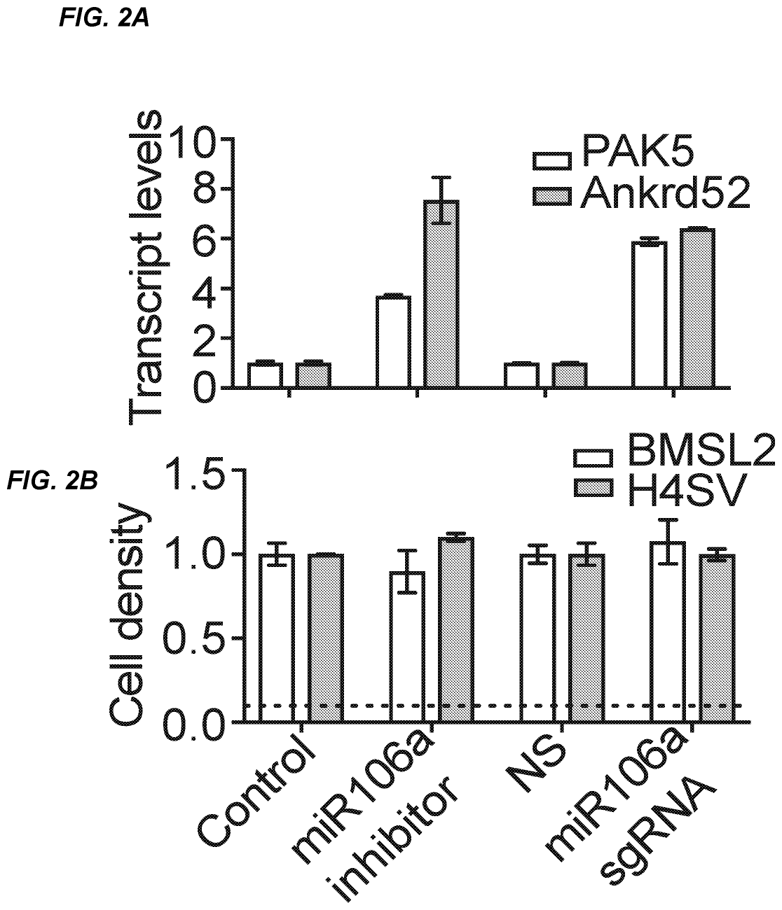

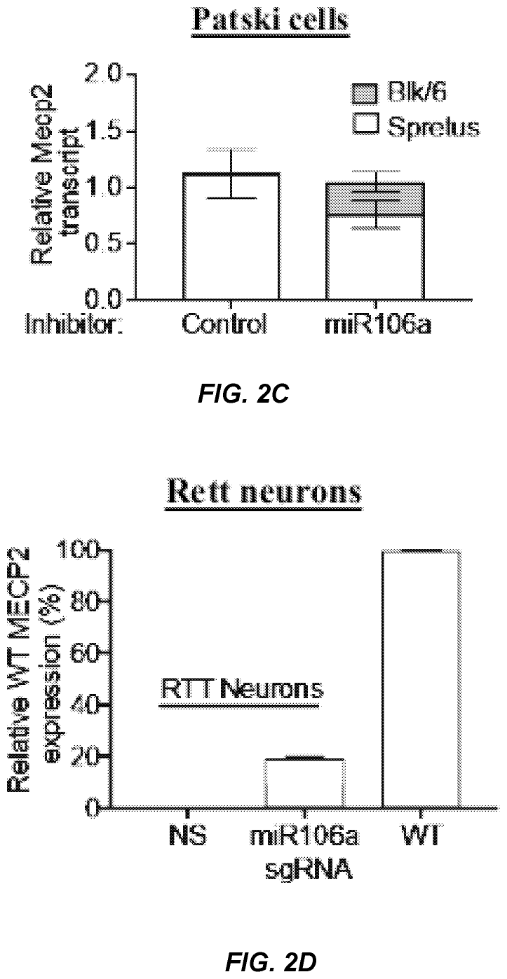

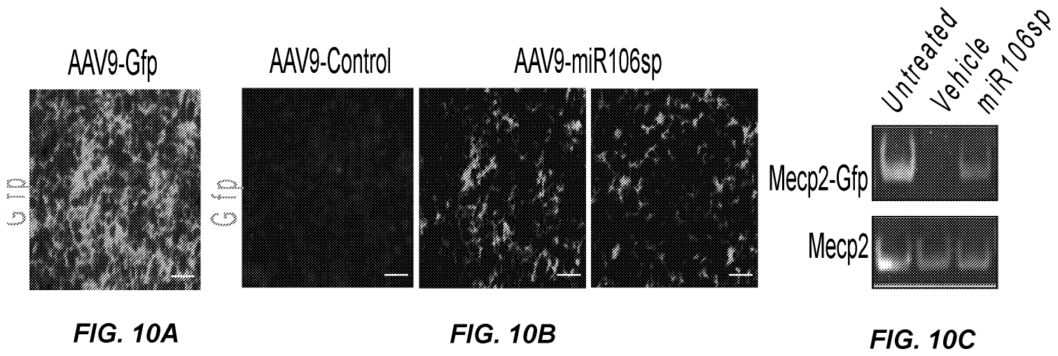

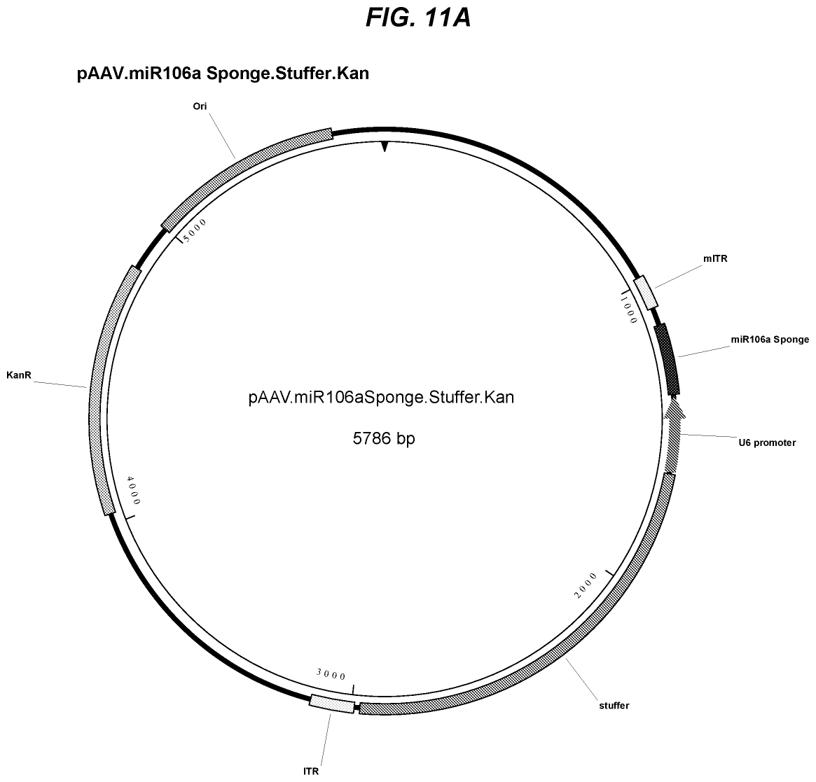

Next it was tested if miR106a inhibition reactivates Xi-linked MECP2 in human post-mitotic neurons, a cell type most relevant to RTT (8, 9). To this end, single-stranded, and chemically enhanced RNA oligonucleotides were utilized to inhibit miR106a. For convenience, these agents are referred herein to as miR106a inhibitor (miR106i). RTT neurons were used that carry T158M missense mutation in MECP2 on active X, but wild type MECP2 gene on Xi (10). Since females are mosaic for XCI, an RTT iPSC clone, derived from the same RTT patient, carrying wild type MECP2 on active X and mutant MECP2 on Xi with complete skewedness to the wild type MECP2 is used as a positive isogenic control (WT-iPSC, (44)). It has been previously shown that WT neurons are phenotypically normal compared to RTT neurons. For example, RTT neurons showed slower growth, smaller soma size and fewer branch points relative to WT neurons as determined by viability and immunofluorescence assays (for example, see (2)). Significantly, miR106i treated RTT neurons expressed Xi-linked MECP2 to the level of ˜12% to that observed in WT neurons (FIG. 10). As expected, miR106a inhibition up-regulated known miR106a targets, PAK5 (11) and Ankrd52 (7); FIG. 2A) but did not affect cell viability (FIG. 2B), indicating that miR106a are target-specific and safe in vitro. Furthermore, inhibition of miR106a with a miR106a inhibitor (FIG. 2C) or miR106a-specific-sgRNA (FIG. 2D) was shown to reactivate MECP2 in Patski cells (FIG. 2C) and Rett neurons (FIG. 2D).

### Example 2

### Mapping of High-Confidence miR106a-Xist Interactions

Given that Xist is a crucial regulatory factor of XCI (12-14) and harbors multiple miR106a seed sequences (7), it was investigated whether miR106a targets Xist. Using computational prediction algorithms (15), five putative binding sites for miR106a were identified in 5′ region of Xist defined as repeat (referred to herein as RepA). Although molecular function of RepA is unclear, RepA-mediated recruitment of proteins, such as RBM15/15b (16) and SPEN (17), is critical for Xist function in XCI.

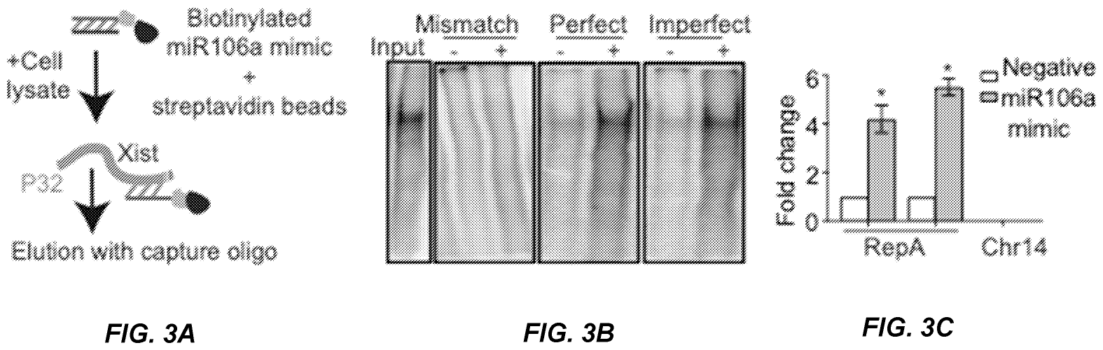

To directly confirm miR106a-RepA interaction, competitive elution of RepA transcript was carried out in complex with biotinylated miR106a mimics (FIG. 3A). In order to substantiate the target specificity of miR106a mimic, a luciferase reporter gene construct was designed expressing a known miR106a target, PAK5 in psi-CHECK-2 reporter system. A ˜80% decrease in luciferase signal was observed compared with the control, which was rescued by the addition of miR106i, confirming the specificity of miR106a mimic and miR106i.

Next the elution efficiency of 5′-P32-radiolabeled RepA transcript was compared using mismatch, perfect, and imperfect complementary capture oligonucleotides for each of the five predicted miR106a binding sites. As shown in a representative subset of results (FIG. 3B), a greater RepA transcript was detected in the pooled washes subsequent to elution with perfect and imperfect complementarity but not with mismatch capture oligonucleotides. Similar analysis was done with full length RepA transcript that confirmed miR106a binding. In conclusion, these results demonstrate that miR106a physically interacts with RepA at multiple sites.

Next, to confirm miR106a binding to endogenous RepA, in-cell pull-down assay using biotinylated miR106a mimic was performed. Biotinylated miRNA/RNA complex from whole cell lysates was extracted using streptavidin beads and analyzed by quantitative RT-PCR (qRT-PCR) for RepA enrichment. A pull-down complex was enriched for RepA in miR106a mimic transfected cells, while no RepA signal was observed in negative control (FIG. 3C), confirming that miR106a and RepA form a complex in vivo.

### Example 3

### MiR106a Transcriptionally Regulates Xist

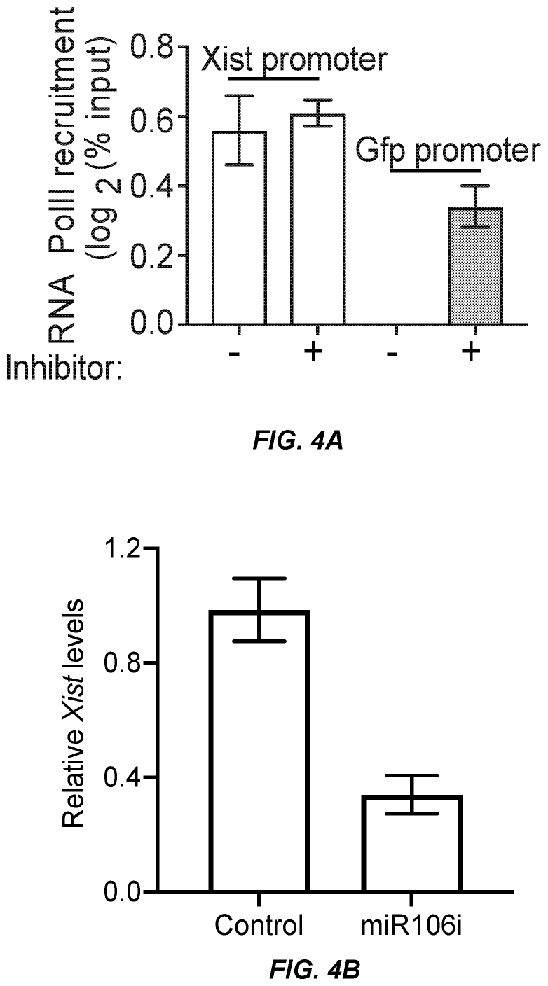

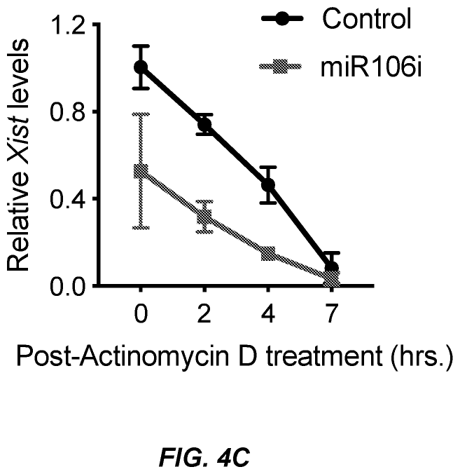

Next it was investigated whether miR106a could positively regulate Xist transcription by either depleting a repressor or indirectly affecting Xist stability. Therefore, RNA polymerase II (PolII) recruitment on Xist promoter by chromatin immunoprecipitation (ChIP) in miR106a-depleted cells was examined. Surprisingly, miR106a depletion did not affect PolII recruitment on Xist promoter but, as expected, Gfp promoter (an Xi-linked transgene in H4SV cells) was enriched for PolII, indicating Xi reactivation (FIG. 4A). miR106a depletion reduced Xist levels (FIG. 4B) and actinomycin D assay showed significantly reduced half life of Xist (FIG. 4C).

### Example 4

### Functional Interaction of miR106a with RepA

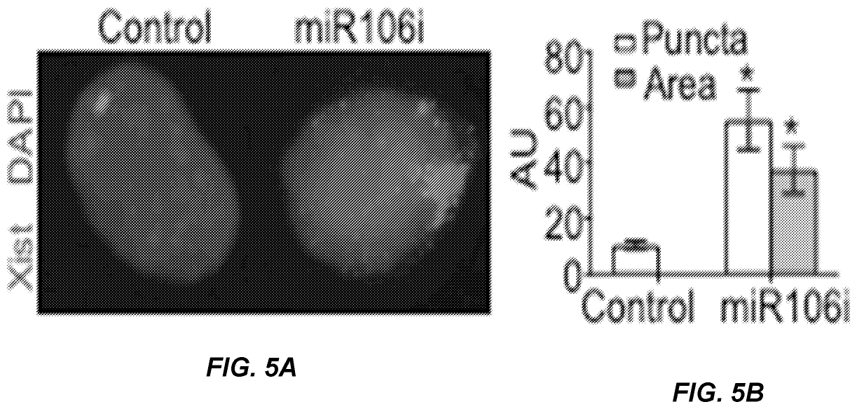

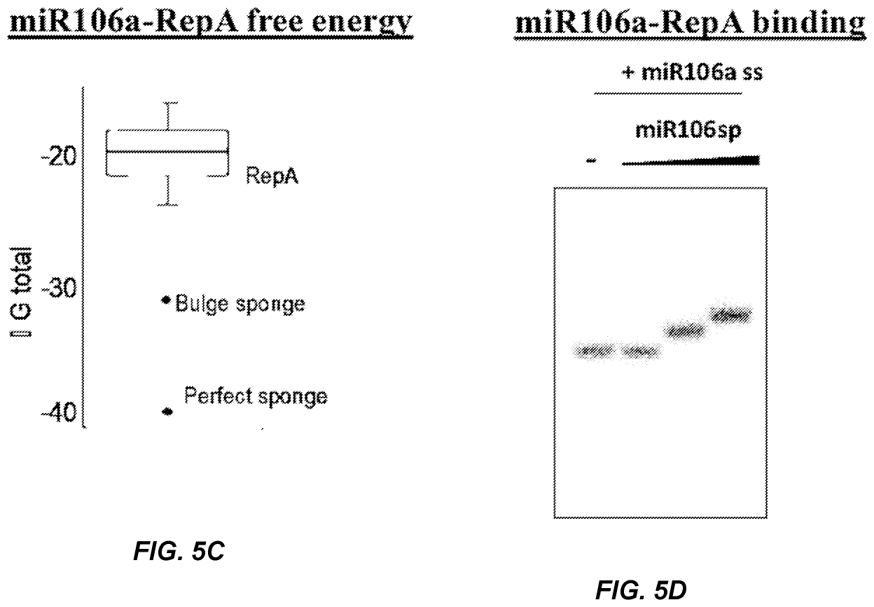

Xist function and its association with Xi is dependent on its structure (18, 36). Therefore, it was investigated whether miR106a depletion affects Xist association with Xi using RNA in situ hybridization (RNA-FISH). As expected, ˜80% of Xist “clouds” were observed in control cells (FIG. 5A, Left). In contrast, depletion of miR106a caused a dramatic change in the Xist “clouds” which appeared dispersed throughout nucleus in ˜65% of cells (number of puncta, FIGS. 5A-B) as well as more diffused on Xi in ˜45% of cells (area of cloud, FIGS. 5A-B). In aggregate, these results suggest that miR106a is crucial for Xist localization to Xi.

### Example 5

### To Determine Whether Inhibition of miR106a can Normalize Dysfunctional Neuronal Phenotypes

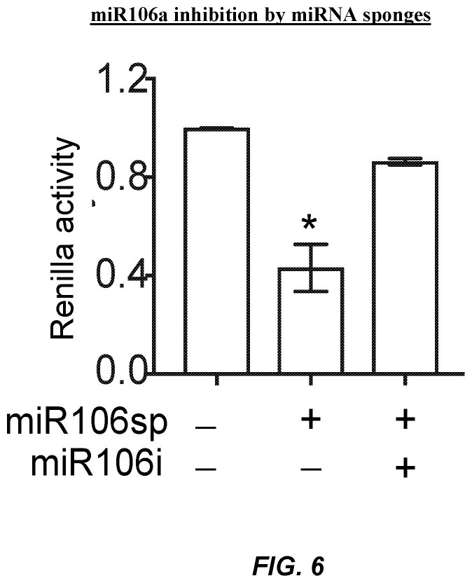

To achieve efficient miR106a inhibition in vivo, a miR106a loss-of-function “sponge” was designed that harbors tandem multiplex of imperfect complementary sequences to the nucleotide sequence of miR106a. This sponge sequence is referred to as miR106sp. It was demonstrated that miR106sp is fully functional based on following parameters; (i) Gibbs free energy: miR106a showed lower ΔGtotal to miR106sp than to RepA region (FIG. 5C). (ii) Functional assay: The miR106sp-miR106a physical association was confirmed by expressing sponge sequences through a dual luciferase reporter system in BMSL2 cells that endogenously express miR106a. Compared to an empty vector, reporter vectors with miR106sp sequences showed ˜60% lower Renilla/Firefly ratio, which is rescued by miR106i (FIG. 6). (iii) Transcriptional effect: miR106sp-mediated sequestration of miR106a reactivates Xi-linked TgGfp and miR106a known targets, PAK5 and Ankrd52, in H4SV cells. (iii) The sponge mRNA, which contains multiple target sites complementary to a miRNA of interest, is a dominant negative method. The sponges interact with the mature miRNA, their effectiveness was unaffected by the clustering of miRNA precursors (FIG. 5D). Together, the results disclosed herein confirmed that miR106sp is biologically active.

To maximize sponge expression and carry out long-term miR106a loss-of-function studies, lentiviral vector pLKO.1 expressing miR106sp (LTV-miR106sp) was engineered. Transduction efficiency of NPCs was optimized by co-transfecting LTV-miR106sp with pLKO.1 expressing Gfp that results in ˜80% transduction efficiency (data not shown). Furthermore, miR106a depletion does not affect neuronal differentiation as indicated by the expression of NPC and neuronal lineage-specific markers.

Whether reactivation of MECP2 by miR106sp can normalize phenotypes of RTT neurons is tested. It is realized that normalization may be partial rather than complete correction but for simplicity the term “normalize” is used to mean partial or complete correction of a phenotype. To assess rescue of RTT neuronal phenotype, RTT-neuronal lines are analyzed using following quantifiable measurements:

(i) Neuronal phenotype: RTT-NPCs are differentiated into neurons for 4, 8, and 12 weeks and wild type MECP2 expression is confirmed and quantitated by allele-specific Taqman assays. Next, different RTT neuronal lines is assayed for soma size, branch points, neuronal network, puncta density, and synaptic formation.

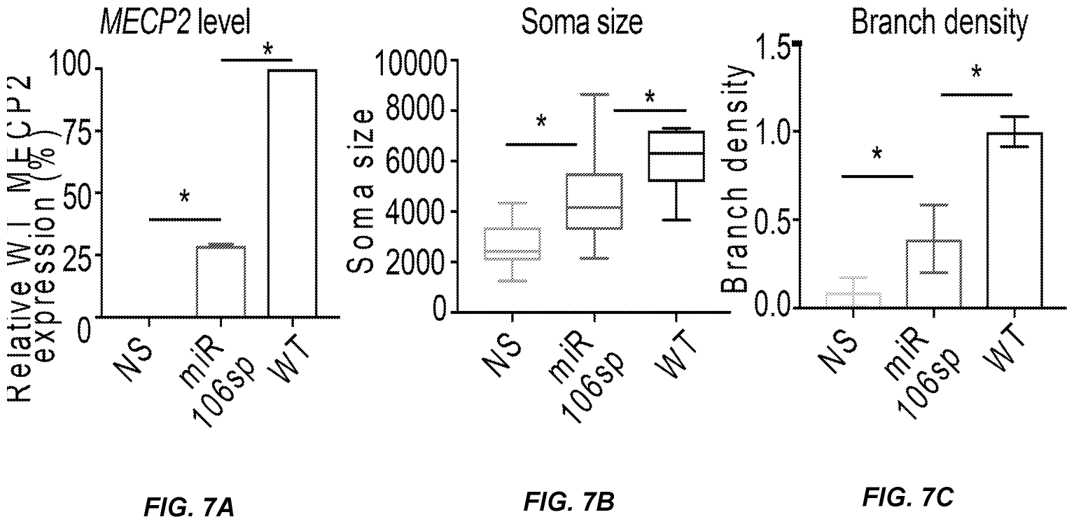

As a proof-of-concept, it was shown that treatment of RTT neurons with LTV-miR106sp expressed wild type MECP2 to the level of ˜30% relative to healthy neurons at ˜8-weeks post-treatment (FIG. 7A) and most significantly, was sufficient to rescue soma size and branch density in MAP2 positive (a neuronal marker) neurons (n=200; FIGS. 7B &7C).

These results: (1) support the hypothesis that even partial reactivation of MECP2 has a normalizing effect on dysfunctional phenotypes of RTT neurons, and (2) demonstrate that the level of MECP2 reactivation achieved has a strong normalizing effect.

(ii) Activity-dependent calcium (Ca²⁺) transients: The spontaneous electrophysiological activity is examined by means of Ca²⁺ imaging in various RTT neuronal lines using gCAMP6s, a Ca²⁺ sensitive fluorescent dye (39). Time-lapse image sequences (63× magnification) are acquired at 28 Hz with a region of 336×256 pixels, using a Zeiss upright fluorescence spin disk confocal microscope. Spontaneous Ca²⁺ transients are analyzed in several independent experiments over time, and images are analyzed by Image J software.

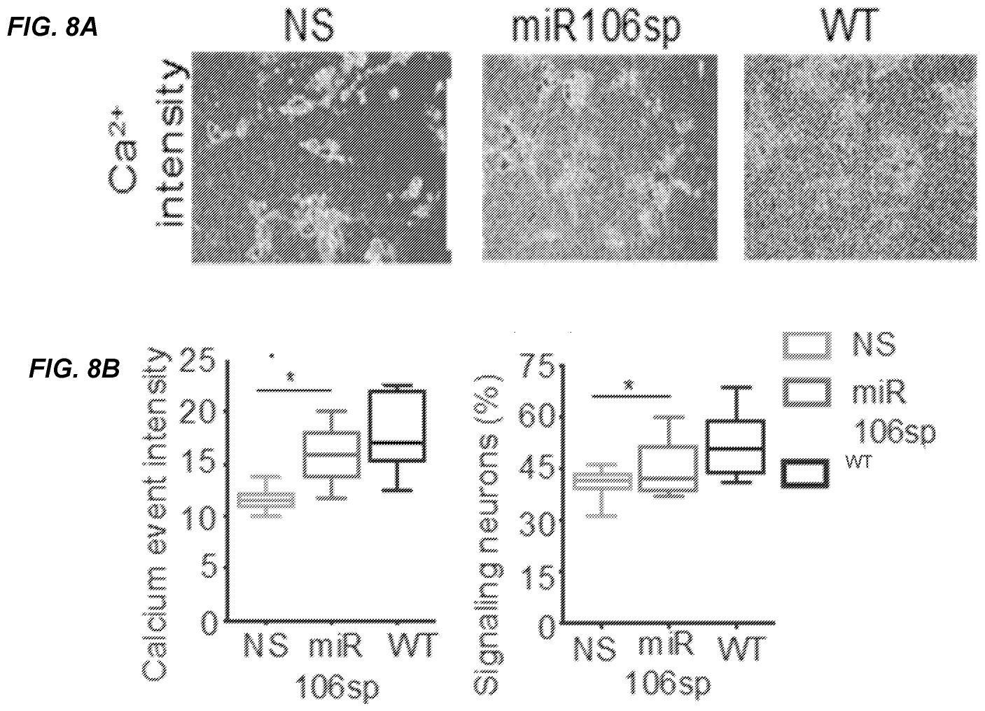

Next activity-dependent Ca²⁺ transients were monitored in LTV-miR106sp treated 8-week old RTT neurons. Briefly, RTT neurons were transduced with GCaMP6s and intracellular Ca²⁺ fluctuations were monitored over time using high-speed imaging. FIG. 8A shows a sharp increase in amplitude and frequency of Ca²⁺ oscillations in miR106sp were depleted but not in control RTT neurons. Notably, Ca²⁺ transient intensity in miR106sp treated cells was comparable to WT neurons (FIG. 8B). While MECP2 expression is optimized following miR106sp treatment, the results suggest miR106a inhibition improved activity-dependent Ca²⁺ transients and also demonstrates feasibility of the proposed approach.

(iii) Excitatory synaptic signaling: The effect of MECP2 restoration on functional maturation of RTT-neurons is determined using electrophysiological methods. Whole cell recordings are performed on neurons that have been differentiated for at least 6 weeks. Changes in frequency and amplitude of spontaneous postsynaptic currents are evaluated in RTT neurons following wild type MECP2 expression.

### Example 6

### Construct of Gene Therapy Constructs

The sponge cassette described in Example 4 (miR106sp) was subcloned into a self-complementary AAV9 genome under U6 promoter. The plasmid construct included a U6 promoter, the miR106a sponge cassette (miR106sp), stuffer sequence, inverted terminal repeats (ITR), mutant ITR (mITR), origin of replication (Ori) and a kanamycin resistance cassette (KanR). A schematic of the plasmid constructs is provided in FIG. 11A referred to as pAAV.miR106a Sponge.Stuffer.Kan. The precise sequence ranges, strand direction and length of the pAAV.miR106a Sponge.Stuffer.Kan components are provided in Table 1. The plasmid sequence of pAAV.miR106a Sponge.Stuffer.Kan is provided in FIG. 11B and is provided in SEQ ID NO: 21. The pAAV.miR106a Sponge.Stuffer.Kan constructs were packaged into an AAV9 genome and expressed accordingly to routine methods known in the art.

TABLE 1

| Name | Range | Strand | Length |
|---|---|---|---|
| mITR | 980 . . . 1085 | [symbol] | 106 |
| MiR106a Sponge | complement | [symbol] | 225 |
| | (1144 . . . 1368) | | |
| U6 promoter | complement | [symbol] | 241 |
| | (1375 . . . 1615) | | |
| stuffer | 1623 . . . 2972 | [symbol] | 1350 |
| ITR | 2991 . . . 3131 | [symbol] | 141 |
| KanR | 4034 . . . 4843 | [symbol] | 810 |
| Ori | 4997 . . . 5618 | [symbol] | 622 |

A short hairpin RNA construct of the mIRNA106a was also generated. The mIRNA106a shRNA was subcloned into a self-complementary AAV9 genome under U6 promoter. The plasmid construct included a U6 promoter, the miR106a shRNA, stuffer sequence, inverted terminal repeats (ITR), mutant ITR (mITR), origin of replication (Ori) and a kanamycin resistance cassette (KanR). A schematic of the plasmid constructs is provided in FIG. 12A referred to as pAAV.miR106a shRNA.Stuffer.Kan. The precise sequence ranges, strand direction and length of the pAAV.miR106a shRNA.Stuffer.Kan components are provided in Table 2. The plasmid sequence of pAAV.miR106a shRNA.Stuffer.Kan is provided in FIG. 12B and is provided in SEQ ID NO: 22. The pAAV.miR106a shRNA.Stuffer.Kan constructs were packaged into an AAV9 genome and expressed accordingly to routine methods known in the art. The efficient adeno-associated virus serotype 9 (AAV9) vector-expressing miR106sp was referred as AAV9-miR106sp. The miR106sp expression, driven by the U6 promoter, was packaged in a self-complementary AAV9 vector. The expression cassette also contained a stuffer to ensure optimal size for packaging (40, 41).

TABLE 2

| Name | Range | Strand | Length |
|---|---|---|---|
| mITR | 980 . . . 1085 | [symbol] | 106 |
| MiR106a shRNA | complement | [symbol] | 56 |
| | (1144 . . . 1199) | | |
| U6 promoter | complement | [symbol] | 241 |
| | (1206 . . . 1446) | | |
| stuffer | 1454 . . . 2803 | [symbol] | 1350 |
| ITR | 2822 . . . 2962 | [symbol] | 141 |
| KanR | 3865 . . . 4674 | [symbol] | 810 |
| Ori | 4828 . . . 5449 | [symbol] | 622 |

### Example 7

### To Determine Whether Inhibition of miR106a can Normalize Behavioral Deficit in Female ACpG RTT Preclinical Models

As described in example 5, an efficient AAV9 vector-expressing miR106sp (referred as AAV9-miR106sp) was engineered to investigate inhibition of miR106a in vivo. As a negative control, empty viral particles were used (AAV9-control). AAV9-miR106sp particles were produced using a triple-transfection method with the transfer and helper plasmids (42). Viral vector concentration was determined by silvergel and Taqman qRT-PCR.

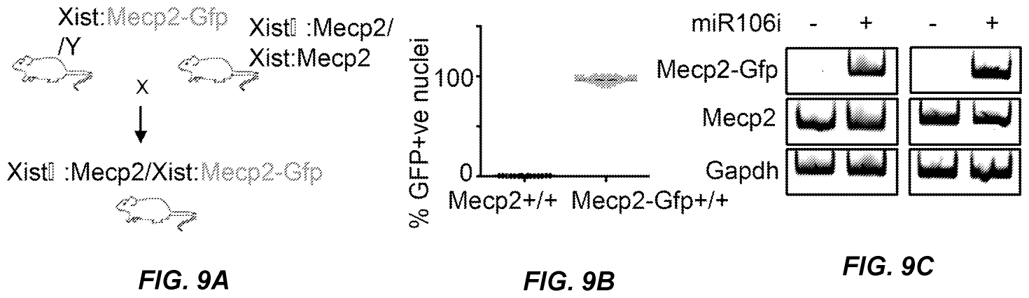

Next it was tested if AAV9-mir106sp reactivates MECP2 in the brain of XistΔ:Mecp2/Xist:Mecp2-Gfp mice (2, 3). More recently XCI mouse model, by crossing Xist:Mecp2-Gfp/Y mouse with XistΔ:Mecp2/Xist:Mecp2 mouse (FIG. 9A, (2)). It was demonstrated that this model allows accurate and robust quantitation of Xi-linked Mecp2 reactivation primarily for two reasons; (i) the results are not precluded by the mosaic expression of GFP with 100% cells carrying Mecp2-Gfp on Xi. Importantly, a FACS-based approach was established and showed that all the cortical nuclei from XistΔ:Mecp2/Xist:Mecp2 are Gfp negative, while 100% of the nuclei from Xist:Mecp2-Gfp/Xist:Mecp2-Gfp are Gfp positive, which represent the theoretical maximum in the experiment (FIG. 9B). (ii) the genetic labeling of Mecp2 permits direct visualization of individual neurons with Gfp, thereby minimizing the experimental manipulations of cells (2). To assess the feasibility of the XistΔ:Mecp2/Xist:Mecp2-Gfp mouse model for monitoring Xi-linked Mecp2 de-repression and treated mouse embryonic fibroblasts isolated from female XistΔ:Mecp2/Xist:Mecp2-Gfp embryos (d15.5) with either control or miR106i. miR106i treatment, but not control, de-repressed Xi-Mecp2-Gfp (FIG. 9C).

Next, a single dose of 5.0e+10 vector genome/kg AAV9-miR106sp or AAV9-control, in neonatal were administered through ICV route as previously described (42). Using AAV9 expressing Gfp (AAV9-Gfp), it was confirmed efficient transduction efficiency of AAV9 vector and demonstrated uniform distribution in the brain of XistΔ:Mecp2/Xist:Mecp2 mice (n=2; FIG. 10A). Significantly, Xi-Mecp2-Gfp expression is detected in mice injected with AAV9-miR106sp at 5 weeks but not in AAV9-control injected mice (FIG. 10B). The expression of Mecp2-Gfp in RNA isolated from mouse brain were also confirmed using RT-PCR (FIG. 10C). Notably, in the ongoing experiments in RTT mice, no sign of distress has been observed ˜15 week's post-miR106a inhibition.

Results presented above provide a compelling evidence for the feasibility of AAV9-miR106sp to inhibit miR106a in vivo; strongly support the hypothesis that inhibiting miR106a reactivates Mecp2 from Xi; and suggests that Xi reactivation is well tolerated in vivo.

Optimal dose and CSF delivery of AAV9-miR106sp: Next the most effective dose at which AAV9-miR106sp expresses maximum Xi-linked Mecp2 in XistΔ:Mecp2/Xist:Mecp2-Gfp mice is confirmed using three different concentrations and injection via cerebrospinal fluid. Mice are injected on post-natal day 1 into the CSF using intracerebroventricular injections at doses ranging 1e10 vg, 2.5e10 vg and 5e10 vg per animal. The Mecp2-Gfp expression is quantitated at the RNA level (qRT-PCR) and at the protein level (flow cytometry, immunofluorescence and immunohistochemistry).

### Example 8

### Rescue of Behavioral Deficit and Improved Survival in RTT Model by AAV9-miR106sp

Rescue of behavioral deficit in ΔCpG-RTT model by AAV9-miR106sp: Comprehensive assessment of phenotypic female RTT mouse model is critical for translation of the disclosed therapy to RTT patients. Consequently, rescue of a wide-range of behavioral measures across development were evaluated in AAV9-miR106sp-treated Tsix^{ΔCpG}:Mecp2/Tsix:Mecp2^{null} (ΔCpG-RTT, Proc Natl Acad Sci USA. 2018 Aug. 7; 115(32):8185-8190) female mice on a standard C57BL/6J background. ACpG-RTT female mice are deficient for Tsix and MECP2 on opposite X chromosomes and therefore, null MECP2 allele is preferentially expressed. Treated female mice were scored on symptoms known to arise from MECP2 disruption and presented in RTT patients: motor weakness, increased tremors, gait disturbance, repetitive behaviors, and self-injury (Proc Natl Acad Sci USA. 2018 Aug. 7; 115(32):8185-8190; Hum Mol Genet. 2018 Dec. 1; 27(23): 4077-4093).

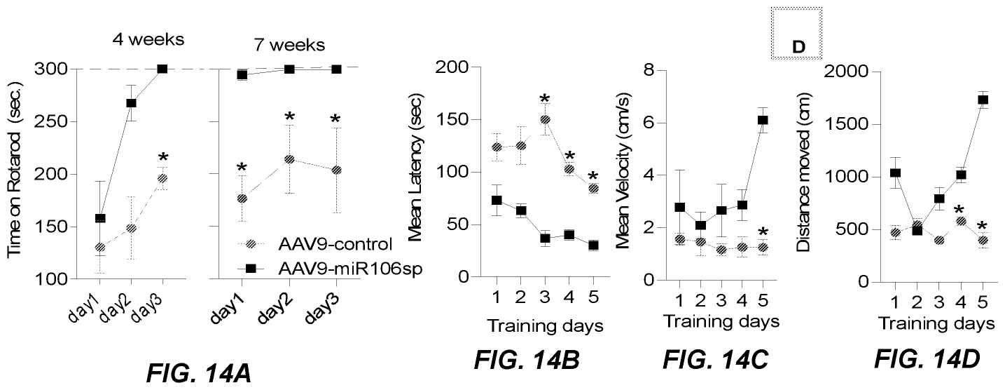

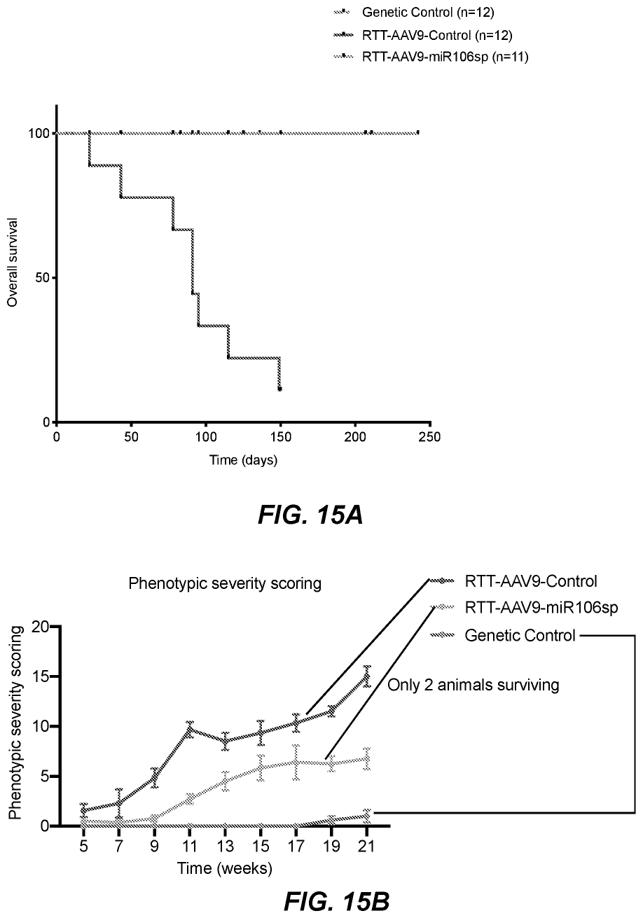

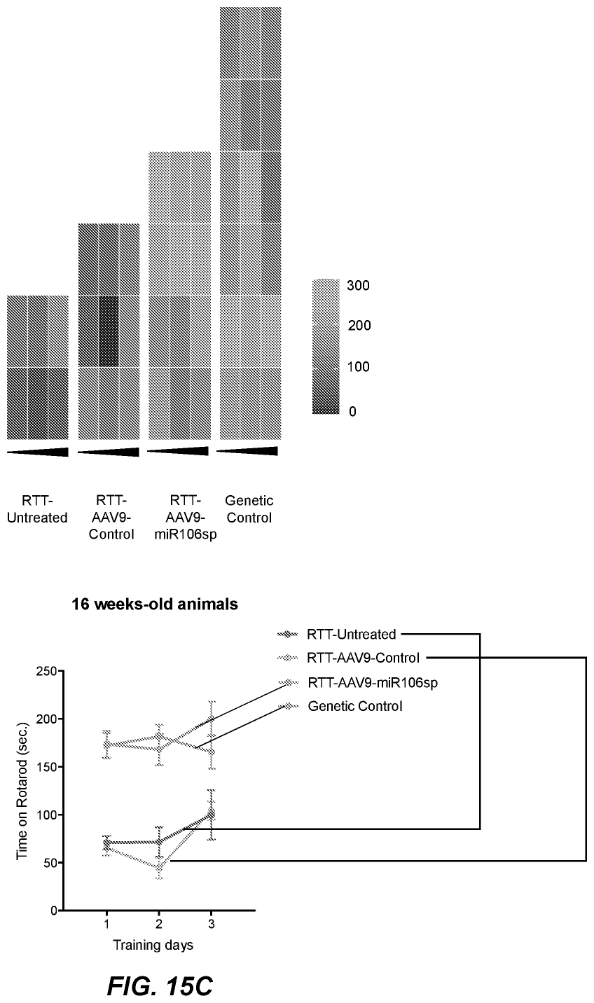

Previous work has identified abnormalities in motor function in MECP2 mutant mice reminiscent of motor impairments observed in RTT girls (Hum Mol Genet. 2018 Dec. 1; 27(23): 4077-4093). Proof-of-concept experiments were performed to assess improvements in motor coordination and learning using the accelerating rotarod (three trials per day, averaged, for three consecutive days). As shown in FIG. 14A, AAV9-miR106sp-injected mice outperformed AAV9-control injected mice on day 2 and 3 at both 4- and 7-weeks. At 7-weeks of age, AAV9-miR106sp-injected mice showed dramatic improvement from a baseline at day 1 compared to the AAV9-control-treated mice, suggesting improvements in motor coordination and learning. These data were also confirmed in mice at 16 weeks of age, whereby AAV9-miR106sp treated mice exhibited strong improvement of rotarod performance compared to AAV9-control or untreated mice (FIG. 15C).

Likewise, on a Barnes Maze (three trials per day, averaged, for five consecutive days at 7-weeks), AAV9-miR106sp treatment produced significant improvements in cognition as evidenced by 1) decreased latency to identify the spatial location of previously rewarded response (FIG. 14B) and 2) increased velocity to complete the response (FIG. 14C). The statistically significant elevations in distance moved during training reveal that treated mice displayed more exploratory behavior and greater reductions in anxiety compared to controls which have high levels of immobility (FIG. 14D). In contrast, AAV9-control-injected mice spent more time in the arena than the AAV9-miR106sp injected mice that was also confirmed in open field exploration test.

The survival and phenotypic severity was also assessed in AAV9.miR106sp treated animals versus controls. As shown in FIG. 15A, AAV9-miR106sp-injected mice showed drastically improved survival up to 250 days compared to AAV9-Control (empty viral particle) treated animals, which displayed survival of around 80-100 days (median survival 91 days). The phenotypic severity of the AAV9.miR106sp treated animals versus controls was also assessed by phenotypic scoring, demonstrating that AAV9.miR106sp treated animals exhibited reduced phenotypic severity up to 21 weeks of age compared to AAV9-Control treated animals.

Together, these preliminary results show that MECP2 restoration through miR106a inhibition rescues neuromotor and learning deficits in ACpG-RTT female mice.

### REFERENCES

1. Neul J L, Zoghbi H Y. Rett syndrome: a prototypical neurodevelopmental disorder. Neuroscientist. 2004; 10(2):118-28. doi: 10.1177/1073858403260995. PubMed PMID: 15070486.
2. Przanowski P, Wasko U, Zheng Z, Yu J, Sherman R, Zhu L J, McConnell M J, Tushir-Singh J, Green M R, Bhatnagar S. Pharmacological reactivation of inactive X-linked Mecp2 in cerebral cortical neurons of living mice. Proc Natl Acad Sci USA. 2018. doi: 10.1073/pnas.1803792115. PubMed PMID: 30012595.
3. Przanowski P, Zheng Z, Wasko U, Bhatnagar S. A Non-random Mouse Model for Pharmacological Reactivation of Mecp2 on the Inactive X Chromosome. J Vis Exp. 2019(147). Epub 2019/06/11. doi: 10.3791/59449. PubMed PMID: 31180354.
4. Yildirim E, Kirby J E, Brown D E, Mercier F E, Sadreyev R I, Scadden D T, Lee J T. Xist RNA is a potent suppressor of hematologic cancer in mice. Cell. 2013; 152(4):727-42. doi: 10.1016/j.cell.2013.01.034. PubMed PMID: 23415223; PMCID: PMC3875356.
5. Komura J, Sheardown S A, Brockdorff N, Singer-Sam J, Riggs A D. In vivo ultraviolet and dimethyl sulfate footprinting of the 5′ region of the expressed and silent Xist alleles. J Biol Chem. 1997; 272(16):10975-80. PubMed PMID: 9099757.
6. Bhatnagar S, Zhu X, Ou J, Lin L, Chamberlain L, Zhu L J, Wajapeyee N, Green M R. Genetic and pharmacological reactivation of the mammalian inactive X chromosome. Proc Natl Acad Sci USA. 2014; 111(35):12591-8. Epub 2014/08/20. doi: 10.1073/pnas.1413620111. PubMed PMID: 25136103; PMCID: Pmc4156765.
7. Imig J, Brunschweiger A, Brummer A, Guennewig B, Mittal N, Kishore S, Tsikrika P, Gerber A P, Zavolan M, Hall J. miR-CLIP capture of a miRNA targetome uncovers a lincRNA H19-miR-106a interaction. Nature chemical biology. 2015; 11(2):107-14. Epub 2014/12/23. doi: 10.1038/nchembio.1713. PubMed PMID: 25531890.
8. Johnston M V, Blue M E, Naidu S. Rett syndrome and neuronal development. J Child Neurol. 2005; 20(9):759-63. Epub 2005/10/18. doi: 10.1177/08830738050200091101. PubMed PMID: 16225832.
9. Johnston M V, Mullaney B, Blue M E. Neurobiology of Rett syndrome. J Child Neurol. 2003; 18(10):688-92. Epub 2003/12/03. doi: 10.1177/08830738030180100501. PubMed PMID: 14649550.
10. Marchetto M C, Carromeu C, Acab A, Yu D, Yeo G W, Mu Y, Chen G, Gage F H, Muotri A R. A model for neural development and treatment of Rett syndrome using human induced pluripotent stem cells. Cell. 2010; 143(4):527-39. doi: 10.1016/j.cell.2010.10.016. PubMed PMID: 21074045; PMCID: PMC3003590.
11. Pan Y J, Wei L L, Wu X J, Huo F C, Mou J, Pei D S. MiR-106a-5p inhibits the cell migration and invasion of renal cell carcinoma through targeting PAK5. Cell Death Dis. 2017; 8(10):e3155. doi: 10.1038/cddis.2017.561. PubMed PMID: 29072688; PMCID: PMC5680926.
12. Arthold S, Kurowski A, Wutz A. Mechanistic insights into chromosome-wide silencing in X inactivation. Human genetics. 2011; 130(2):295-305. Epub 2011/05/14. doi: 10.1007/s00439-011-1002-0. PubMed PMID: 21567178.
13. Pontier D B, Gribnau J. Xist regulation and function explored. Hum Genet. 2011; 130(2):223-36. Epub 2011/06/01. doi: 10.1007/s00439-011-1008-7. PubMed PMID: 21626138; PMCID: PMC3132428.
14. Wutz A. Gene silencing in X-chromosome inactivation: advances in understanding facultative heterochromatin formation. Nat Rev Genet. 2011; 12(8):542-53. Epub 2011/07/19. doi: 10.1038/nrg3035. PubMed PMID: 21765457.
15. Riffo-Campos A L, Riquelme I, Brebi-Mieville P. Tools for Sequence-Based miRNA Target Prediction: What to Choose? Int J Mol Sci. 2016; 17(12). doi: 10.3390/ijms17121987. PubMed PMID: 27941681; PMCID: PMC5187787.
16. Patil D P, Chen C K, Pickering B F, Chow A, Jackson C, Guttman M, Jaffrey S R. m(6)A RNA methylation promotes XIST-mediated transcriptional repression. Nature. 2016; 537(7620):369-73. doi: 10.1038/nature19342. PubMed PMID: 27602518; PMCID: PMC5509218.
17. Lu Z, Zhang Q C, Lee B, Flynn R A, Smith M A, Robinson J T, Davidovich C, Gooding A R, Goodrich K J, Mattick J S, Mesirov J P, Cech T R, Chang H Y. RNA Duplex Map in Living Cells Reveals Higher-Order Transcriptome Structure. Cell. 2016; 165(5):1267-79. doi: 10.1016/j.cell.2016.04.028. PubMed PMID: 27180905; PMCID: PMC5029792.
18. Wutz A, Rasmussen T P, Jaenisch R. Chromosomal silencing and localization are mediated by different domains of Xist RNA. Nat Genet. 2002; 30(2)167-74. doi: 10.1038/ng820. PubMed PMID: 11780141.
19. Duszczyk M M, Wutz A, Rybin V, Sattler M. The Xist RNA A-repeat comprises a novel AUCG tetraloop fold and a platform for multimerization. RNA. 2011; 17(11):1973-82. doi: 10.1261/rna.2747411. PubMed PMID: 21947263; PMCID: PMC3198591.
20. Deigan K E, Li T W, Mathews D H, Weeks K M. Accurate SHAPE-directed RNA structure determination. Proc Natl Acad Sci USA. 2009; 106(1):97-102. doi: 10.1073/pnas.0806929106. PubMed PMID: 19109441; PMCID: PMC2629221.
21. Smola M J, Christy T W, Inoue K, Nicholson C O, Friedersdorf M, Keene J D, Lee D M, Calabrese J M, Weeks K M. SHAPE reveals transcript-wide interactions, complex structural domains, and protein interactions across the Xist lncRNA in living cells. Proc Natl Acad Sci USA. 2016; 113(37):10322-7. doi: 10.1073/pnas.1600008113. PubMed PMID: 27578869; PMCID: PMC5027438.
22. Smola M J, Rice G M, Busan S, Siegfried N A, Weeks K M. Selective 2′-hydroxyl acylation analyzed by primer extension and mutational profiling (SHAPE-MaP) for direct, versatile and accurate RNA structure analysis. Nat Protoc. 2015; 10(11):1643-69. doi: 10.1038/nprot.2015.103. PubMed PMID: 26426499; PMCID: PMC4900152.
23. Duncan C D, Weeks K M. SHAPE analysis of long-range interactions reveals extensive and thermodynamically preferred misfolding in a fragile group I intron RNA. Biochemistry. 2008; 47(33):8504-13. doi: 10.1021/bi800207b. PubMed PMID: 18642882; PMCID: PMC4900158.
24. Gherghe C M, Shajani Z, Wilkinson K A, Varani G, Weeks K M. Strong correlation between SHAPE chemistry and the generalized NMR order parameter (S2) in RNA. J Am Chem Soc. 2008; 130(37):12244-5. doi: 10.1021/ja804541s. PubMed PMID: 18710236; PMCID: PMC2712629.
25. Grohman J K, Kottegoda S, Gorelick R J, Allbritton N L, Weeks K M. Femtomole SHAPE reveals regulatory structures in the authentic XMRV RNA genome. J Am Chem Soc. 2011; 133(50):20326-34. doi: 10.1021/ja2070945. PubMed PMID: 22126209; PMCID: PMC3241870.
26. Hajdin C E, Bellaousov S, Huggins W, Leonard C W, Mathews D H, Weeks K M. Accurate SHAPE-directed RNA secondary structure modeling, including pseudoknots. Proc Natl Acad Sci USA. 2013; 110(14):5498-503. doi: 10.1073/pnas.1219988110. PubMed PMID: 23503844; PMCID: PMC3619282.
27. Lavender C A, Lorenz R, Zhang G, Tamayo R, Hofacker I L, Weeks K M. Model-Free RNA Sequence and Structure Alignment Informed by SHAPE Probing Reveals a Conserved Alternate Secondary Structure for 16S rRNA. PLoS Comput Biol. 2015; 11(5):e1004126. doi: 10.1371/journal.pcbi.1004126. PubMed PMID: 25992778; PMCID: PMC4438973.
28. Leonard C W, Hajdin C E, Karabiber F, Mathews D H, Favorov O V, Dokholyan N V, Weeks K M. Principles for understanding the accuracy of SHAPE-directed RNA structure modeling. Biochemistry. 2013; 52(4):588-95. doi: 10.1021/bi300755u. PubMed PMID: 23316814; PMCID: PMC3578230.
29. Low J T, Knoepfel S A, Watts J M, ter Brake O, Berkhout B, Weeks K M. SHAPE-directed discovery of potent shRNA inhibitors of HIV-1. Mol Ther. 2012; 20(4):820-8. doi: 10.1038/mt.2011.299. PubMed PMID: 22314289; PMCID: PMC3321596.
30. Low J T, Weeks K M. SHAPE-directed RNA secondary structure prediction. Methods. 2010; 52(2):150-8. doi: 10.1016/j.ymeth.2010.06.007. PubMed PMID: 20554050; PMCID: PMC2941709.
31. McGinnis J L, Duncan C D, Weeks K M. High-throughput SHAPE and hydroxyl radical analysis of RNA structure and ribonucleoprotein assembly. Methods Enzymol. 2009; 468:67-89. doi: 10.1016/S0076-6879(09)68004-6. PubMed PMID: 20946765; PMCID: PMC4890575.
32. McGinnis J L, Dunkle J A, Cate J H, Weeks K M. The mechanisms of RNA SHAPE chemistry. J Am Chem Soc. 2012; 134(15):6617-24. doi: 10.1021/ja2104075. PubMed PMID: 22475022; PMCID: PMC4337229.
33. McGinnis J L, Liu Q, Lavender C A, Devaraj A, McClory S P, Fredrick K, Weeks K M. In-cell SHAPE reveals that free 30S ribosome subunits are in the inactive state. Proc Natl Acad Sci USA. 2015; 112(8):2425-30. doi: 10.1073/pnas.1411514112. PubMed PMID: 25675474; PMCID: PMC4345610.
34. Merino E J, Wilkinson K A, Coughlan J L, Weeks K M. RNA structure analysis at single nucleotide resolution by selective 2′-hydroxyl acylation and primer extension (SHAPE). J Am Chem Soc. 2005; 127(12):4223-31. doi: 10.1021/ja043822v. PubMed PMID: 15783204.
35. Smola M J, Calabrese J M, Weeks K M. Detection of RNA-Protein Interactions in Living Cells with SHAPE. Biochemistry. 2015; 54(46):6867-75. doi: 10.1021/acs.biochem.5b00977. PubMed PMID: 26544910; PMCID: PMC4900165.
36. Maenner S, Blaud M, Fouillen L, Savoye A, Marchand V, Dubois A, Sanglier-Cianferani S, Van Dorsselaer A, Clerc P, Avner P, Visvikis A, Branlant C. 2-D structure of the A region of Xist RNA and its implication for PRC2 association. PLoS Biol. 2010; 8(1):e1000276. doi: 10.1371/journal.pbio.1000276. PubMed PMID: 20052282; PMCID: PMC2796953.
37. Mavrakis K J, Wolfe A L, Oricchio E, Palomero T, de Keersmaecker K, McJunkin K, Zuber J, James T, Khan A A, Leslie C S, Parker J S, Paddison P J, Tam W, Ferrando A, Wendel H G. Genome-wide RNA-mediated interference screen identifies miR-19 targets in Notch-induced T-cell acute lymphoblastic leukaemia. Nat Cell Biol. 2010; 12(4):372-9. doi: 10.1038/ncb2037. PubMed PMID: 20190740; PMCID: PMC2989719.
38. Mu P, Han Y C, Betel D, Yao E, Squatrito M, Ogrodowski P, de Stanchina E, D'Andrea A, Sander C, Ventura A. Genetic dissection of the miR-1792 cluster of microRNAs in Myc-induced B-cell lymphomas. Genes Dev. 2009; 23(24):2806-11. doi: 10.1101/gad.1872909. PubMed PMID: 20008931; PMCID: PMC2800095.
39. Chen T W, Wardill T J, Sun Y, Pulver S R, Renninger S L, Baohan A, Schreiter E R, Kerr R A, Orger M B, Jayaraman V, Looger L L, Svoboda K, Kim D S. Ultrasensitive fluorescent proteins for imaging neuronal activity. Nature. 2013; 499(7458):295-300. doi: 10.1038/nature12354. PubMed PMID: 23868258; PMCID: PMC3777791.
40. Sinnett S E, Hector R D, Gadalla K K E, Heindel C, Chen D, Zaric V, Bailey M E S, Cobb S R, Gray S J. Improved MECP2 Gene Therapy Extends the Survival of MeCP2-Null Mice without Apparent Toxicity after Intracisternal Delivery. Mol Ther Methods Clin Dev. 2017; 5:106-15. doi: 10.1016/j.omtm.2017.04.006. PubMed PMID: 28497072; PMCID: PMC5424572.
41. lannitti T, Scarrott J M, Likhite S, Coldicott IRP, Lewis K E, Heath P R, Higginbottom A, Myszczynska M A, Milo M, Hautbergue G M, Meyer K, Kaspar B K, Ferraiuolo L, Shaw P J, Azzouz M. Translating SOD1 Gene Silencing toward the Clinic: A Highly Efficacious, Off-Target-free, and Biomarker-Supported Strategy for fALS. Mol Ther Nucleic Acids. 2018; 12:75-88. Epub 2018 Sep. 10. doi: 10.1016/j.omtn.2018.04.015. PubMed PMID: 30195799; PMCID: PMC6023790.
42. Foust K D, Salazar D L, Likhite S, Ferraiuolo L, Ditsworth D, Ilieva H, Meyer K, Schmelzer L, Braun L, Cleveland D W, Kaspar B K. Therapeutic AAV9-mediated suppression of mutant SOD1 slows disease progression and extends survival in models of inherited ALS. Mol Ther. 2013; 21(12):2148-59. Epub 2013/09/07. doi: 10.1038/mt.2013.211. PubMed PMID: 24008656; PMCID: PMC3863799.
43. Carrette L L G, Blum R, Ma W, Kelleher R J, 3rd, Lee J T. Tsix-Mecp2 female mouse model for Rett syndrome reveals that low-level MECP2 expression extends life and improves neuromotor function. Proc Natl Acad Sci USA. 2018; 115(32):8185-90. Epub 2018 Jul. 25. doi: 10.1073/pnas.1800931115. PubMed PMID: 30038001; PMCID: PMC6094149.
44. Guy J, Hendrich B, Holmes M, Martin J E, Bird A. A mouse Mecp2-null mutation causes neurological symptoms that mimic Rett syndrome. Nat Genet. 2001; 27(3):322-6. Epub 2001 Mar. 10. doi: 10.1038/85899. PubMed PMID: 11242117.
45. Guy J, Gan J, Selfridge J, Cobb S, Bird A. Reversal of neurological defects in a mouse model of Rett syndrome. Science (New York, N.Y.). 2007; 315(5815):1143-7. Epub 2007 Feb. 10. doi: 10.1126/science.1138389. PubMed PMID: 17289941.
46. Hofseth L J. Getting rigorous with scientific rigor. Carcinogenesis. 2018; 39(1):21-5. Epub 2017 Oct. 3. doi: 10.1093/carcin/bgx085. PubMed PMID: 28968787; PMCID: PMC5862244.
47. Samsa G, Samsa L. A Guide to Reproducibility in Preclinical Research. Acad Med. 2019; 94(1):47-52. Epub 2018 Jul. 12. doi: 10.1097/ACM.0000000000002351. PubMed PMID: 29995667; PMCID: PMC6314499.

We claim:

1. A polynucleotide comprising a microRNA sponge cassette, wherein the microRNA sponge cassette comprises one or more nucleotide sequences that target one or more miRNA of interest.

2. The polynucleotide of claim 1, wherein one or more nucleotide sequences that target the microRNA of interest is a tandem multiplexes of perfectly or imperfectly complementary sequences to the microRNA of interest.

3. The polynucleotide of claim 1, wherein one or more nucleotide sequences that target the microRNA of interest is at least at least 85% complementary to the mature microRNA of interest sequence, at least 90% complementary to the mature microRNA of interest sequence, at least 95% complementary to the mature microRNA of interest sequence, at least 96% complementary to the mature microRNA of interest sequence, at least 97% complementary to the mature microRNA of interest sequence, at least 98% complementary to the mature microRNA of interest sequence or at least 99% complementary to the mature microRNA of interest sequence.

4. The polynucleotide of any one of claims 1-3, wherein the microRNA sponge cassette comprises at least 2 or more nucleotide sequences that target one or more miRNA of interest, at least 3 or more nucleotide sequences that target one or more miRNA of interest, at least 4 or more nucleotide sequences that target one or more miRNA of interest or at least 2 or more nucleotide sequences that target one or more miRNA of interest.

5. The polynucleotide of any one of claims 1-4, wherein the microRNA sponge cassette comprises 2, 4, 6, or 8 repeats of a nucleotide sequences that target the microRNA of interest.

6. The polynucleotide of any one of claims 1-5, wherein the microRNA sponge cassette comprises one or more nucleotide sequences that target miR106a.

7. The polynucleotide of any one of claims 1-6, wherein the nucleotide sequence that targets miRNA of interest comprises the nucleotide sequence of SEQ ID NO: 1 or 2.

8. The polynucleotide of any one of claims 1-7, wherein the microRNA sponge cassette comprises the nucleotide sequence of SEQ ID NO: 3, SEQ ID NO: 4, SEQ ID NO: 5, SEQ ID NO: 6, SEQ ID NO: 7 or SEQ ID NO: 8.

9. A recombinant AAV (rAAV) having a genome comprising the polynucleotide sequence of any one of claims 1-11.

10. The rAAV of claim 9, wherein the genome comprises the U6 or H1 promoter.

11. The rAAV of claim 9 or 10, wherein the genome further comprises a stuffer sequence.

12. The rAAV of claim 11, wherein the stuffer sequence comprises the nucleotide sequence of SEQ ID NO: 11.

13. The rAAV of anyone of claims 9-12, wherein the genome comprises nucleotides 980 to 3131 of the nucleotide sequences of SEQ ID NO: 21.

14. The rAAV of anyone of claims 9-13, wherein the vector is a serotype AAV1, AAV2, AAV3, AAV4, AAV5, AAV6, AAV7, AAV8, AAV9, AAVRH10, AAVRH74, AAV11, AAV12, AAV13, Anc80, or AAV7m8 or their derivatives.

15. A rAAV particle comprising the rAAV of any one of claims 9-14.

16. A composition comprising a polynucleotide of any one of claims 1-8, a rAAV of any one of claims 9-14, or a rAAV particle of claim 15.

17. A method of treating Rett syndrome comprising administering a therapeutically effective amount of the rAAV of any one of claims 9-14, a rAAV particle of claim 15, or the composition of claim 16.

18. A method of activating expression of a X-linked gene comprising administering a therapeutically effective amount of the a rAAV of any one of claims 9-14, a rAAV particle of claim 15, or the composition of claim 16.

19. The method of claim 18, wherein the X-linked gene is Methyl CpG binding protein 2 (MECP2).

20. A method of treating a X-linked disorder comprising administering a therapeutically effective amount of the rAAV of any one of claims 9-14, a rAAV particle of claim 15, or the composition of claim 16.

21. The method of claim 20, wherein the X-linked disorder is rett syndrome, hemophilia A, hemophilia B, Dent's disease 1, Dent's disease 2, DDX3X syndrome, Albinism-deafness syndrome, Aldrich syndrome, Alport syndrome, Anaemia (hereditary hypochromic), Anemia, (sideroblastic with ataxia), Cataract, Charcot-Marie-Tooth, Color blindness, Diabetes (insipidus, nephrogenic), Dyskeratosis congenita, Ectodermal dysplasia, Faciogenital dysplasia, Fabry disease, Glucose-6-phosphate dehydrogenase deficiency, Glycogen storage disease type VIII, Gonadal dysgenesis, Testicular feminization syndrome, Addison's disease with cerebral sclerosis, Adrenal hypoplasia, Granulomatous disease, siderius X-linked mental retardation syndrome, Agammaglobulinaemia Bruton type, Choroidoretinal degeneration, Choroidaemia, Albinism (ocular), fragile X syndrome, Epileptic encephalopathy (early infantile 2), Hydrocephalus (aqueduct stenosis), Hypophosphataemic rickets, Lesch-Nyhan syndrome (hypoxanthine-guanine-phosphoribosyl transferase deficiency), incontinentia pigmenti, Kallmann syndrome, paroxysmal nocturnal hemoglobinuria, Spinal muscular atrophy 2, Spastic paraplegia, Keratosis follicularis spinulosa, Lowe (oculocerebrorenal) syndrome, Menkes syndrome, Renpenning Syndrome, Mental retardation, Coffin-Lowry syndrome, Microphthalmia (Lenz syndrome), Muscular dystrophy (Becker, Duchenne and Emery-Dreifuss types), Myotubular myopathy, Night blindness, Norrie's disease (pseudoglioma), Nystagmus, Orofaciodigital syndrome, Ornithine transcarbamylase deficiency (type I hyperammonaemia), Phosphoglycerate kinase deficiency, Phosphoribosylpyrophosphate synthetase deficiency, Retinitis pigmentosa, Retinoschisis, Muscular atrophy/Dihydrotestosterone receptor deficiency, Spinal muscular atrophy, Spondyloepiphyseal dysplasia tarda, Thrombocytopenia, Thyroxine-binding globulin, McLeod syndrome.

22. Use of a therapeutically effective amount of the a rAAV of any one of claims 9-14, an rAAV particle of claim 15, or the composition of claim 16, for the preparation of a medicament for treating Rett Syndrome.

23. Use of a therapeutically effective amount of the a rAAV of any one of claims 9-14, an rAAV particle of claim 15, or the composition of claim 16, for the preparation of a medicament for activating expression of a X-linked gene.

24. The use of claim 23, wherein the X-linked gene is Methyl CpG binding protein 2 (MECP2).

25. Use of a therapeutically effective amount of the a rAAV of any one of claims 9-14, an rAAV particle of claim 15, or the composition of claim 16, for the preparation of a medicament for the treatment of a X-linked disorder.

26. The use of claim 25, wherein the X-linked disorder is rett syndrome, hemophilia A, hemophilia B, Dent's disease 1, Dent's disease 2, DDX3X syndrome, Albinism-deafness syndrome, Aldrich syndrome, Alport syndrome, Anaemia (hereditary hypochromic), Anemia, (sideroblastic with ataxia), Cataract, Charcot-Marie-Tooth, Color blindness, Diabetes (insipidus, nephrogenic), Dyskeratosis congenita, Ectodermal dysplasia, Faciogenital dysplasia, Fabry disease, Glucose-6-phosphate dehydrogenase deficiency, Glycogen storage disease type VIII, Gonadal dysgenesis, Testicular feminization syndrome, Addison's disease with cerebral sclerosis, Adrenal hypoplasia, Granulomatous disease, siderius X-linked mental retardation syndrome, Agammaglobulinaemia Bruton type, Choroidoretinal degeneration, Choroidaemia, Albinism (ocular), fragile X syndrome, Epileptic encephalopathy (early infantile 2), Hydrocephalus (aqueduct stenosis), Hypophosphataemic rickets, Lesch-Nyhan syndrome (hypoxanthine-guanine-phosphoribosyl transferase deficiency), incontinentia pigmenti, Kallmann syndrome, paroxysmal nocturnal hemoglobinuria, Spinal muscular atrophy 2, Spastic paraplegia, Keratosis follicularis spinulosa, Lowe (oculocerebrorenal) syndrome, Menkes syndrome, Renpenning Syndrome, Mental retardation, Coffin-Lowry syndrome, Microphthalmia (Lenz syndrome), Muscular dystrophy (Becker, Duchenne and Emery-Dreifuss types), Myotubular myopathy, Night blindness, Norrie's disease (pseudoglioma), Nystagmus, Orofaciodigital syndrome, Ornithine transcarbamylase deficiency (type I hyperammonaemia), Phosphoglycerate kinase deficiency, Phosphoribosylpyrophosphate synthetase deficiency, Retinitis pigmentosa, Retinoschisis, Muscular atrophy/Dihydrotestosterone receptor deficiency, Spinal muscular atrophy, Spondyloepiphyseal dysplasia tarda, Thrombocytopenia, Thyroxine-binding globulin, McLeod syndrome.

27. A composition comprising a therapeutically effective amount of the rAAV of any one of claims 9-14, the rAAV particle of claim 15, or the composition of claim 16 for treating Rett Syndrome.

28. A composition comprising a therapeutically effective amount of the rAAV of any one of claims 9-14, the rAAV particle of claim 15, or the composition of claim 16 for activating expression of a X-linked gene.

29. The composition of claim 28, wherein the X-linked gene is Methyl CpG binding protein 2 (MECP2).

30. A composition comprising a therapeutically effective amount of the rAAV of any one of claims 9-14, the rAAV particle of claim 15, or the composition of claim 16 for treating a X-linked disorder.

31. The composition of claim 30, wherein the X-linked disorder is rett syndrome, hemophilia A, hemophilia B, Dent's disease 1, Dent's disease 2, DDX3X syndrome, Albinism-deafness syndrome, Aldrich syndrome, Alport syndrome, Anaemia (hereditary hypochromic), Anemia, (sideroblastic with ataxia), Cataract, Charcot-Marie-Tooth, Color blindness, Diabetes (insipidus, nephrogenic), Dyskeratosis congenita, Ectodermal dysplasia, Faciogenital dysplasia, Fabry disease, Glucose-6-phosphate dehydrogenase deficiency, Glycogen storage disease type VIII, Gonadal dysgenesis, Testicular feminization syndrome, Addison's disease with cerebral sclerosis, Adrenal hypoplasia, Granulomatous disease, siderius X-linked mental retardation syndrome, Agammaglobulinaemia Bruton type, Choroidoretinal degeneration, Choroidaemia, Albinism (ocular), fragile X syndrome, Epileptic encephalopathy (early infantile 2), Hydrocephalus (aqueduct stenosis), Hypophosphataemic rickets, Lesch-Nyhan syndrome (hypoxanthine-guanine-phosphoribosyl transferase deficiency), incontinentia pigmenti, Kallmann syndrome, paroxysmal nocturnal hemoglobinuria, Spinal muscular atrophy 2, Spastic paraplegia, Keratosis follicularis spinulosa, Lowe (oculocerebrorenal) syndrome, Menkes syndrome, Renpenning Syndrome, Mental retardation, Coffin-Lowry syndrome, Microphthalmia (Lenz syndrome), Muscular dystrophy (Becker, Duchenne and Emery-Dreifuss types), Myotubular myopathy, Night blindness, Norrie's disease (pseudoglioma), Nystagmus, Orofaciodigital syndrome, Ornithine transcarbamylase deficiency (type I hyperammonaemia), Phosphoglycerate kinase deficiency, Phosphoribosylpyrophosphate synthetase deficiency, Retinitis pigmentosa, Retinoschisis, Muscular atrophy/Dihydrotestosterone receptor deficiency, Spinal muscular atrophy, Spondyloepiphyseal dysplasia tarda, Thrombocytopenia, Thyroxine-binding globulin, McLeod syndrome.
